SEQUENCE LISTING (16 sequences)

SEQ ID NO: 1 - DNA, 20 bp, Artificial Sequence
  Synthesized
cataggaagctgggagcaag

SEQ ID NO: 2 - DNA, 20 bp, Artificial Sequence
  Synthesized
gccctccaatcagtcttctg

SEQ ID NO: 3 - DNA, 20 bp, Artificial Sequence
  Synthesized
agtggatctgccaactactc

SEQ ID NO: 4 - DNA, 22 bp, Artificial Sequence
  Synthesized
catctacaaacgggaatgtctg

SEQ ID NO: 5 - DNA, 20 bp, Artificial Sequence
  Synthesized
gaggtcaaacccaacccagt

SEQ ID NO: 6 - DNA, 22 bp, Artificial Sequence
  Synthesized
tgttaaggtactgaagcgaggg

SEQ ID NO: 7 - DNA, 20 bp, Artificial Sequence
  Synthesized
tgtgtggggtcctgtcacta

SEQ ID NO: 8 - DNA, 20 bp, Artificial Sequence
  Synthesized
cagtagctgcagggcgtatt

SEQ ID NO: 9 - DNA, 22 bp, Artificial Sequence
  Synthesized
tgctgcagcggctttgaagaag

SEQ ID NO: 10 - DNA, 24 bp, Artificial Sequence
  Synthesized
gcagggtccttcagtttacctgtg

SEQ ID NO: 11 - DNA, 20 bp, Artificial Sequence
  Synthesized
gagcctggcactccatcgaa

SEQ ID NO: 12 - DNA, 20 bp, Artificial Sequence
  Synthesized
ccctgtagtagtgggcgatg

SEQ ID NO: 13 - DNA, 19 bp, Artificial Sequence
  Synthesized
cctcatttgcgccttgcag

SEQ ID NO: 14 - DNA, 20 bp, Artificial Sequence
  Synthesized
ctccgggagatgaaaaacca

SEQ ID NO: 15 - DNA, 23 bp, Artificial Sequence
  Synthesized
agaatgaccacaacctcttcagt

SEQ ID NO: 16 - DNA, 20 bp, Artificial Sequence
  Synthesized
ccttgtggaggttccttgag

(19) United States
(12) Patent Application Publication
(10) Pub. No.: US 2022/0289686 A1
(43) Pub. Date: Sep. 15, 2022

(54) COMPOSITIONS OF NRF2 INHIBITING AGENTS AND METHODS OF USE THEREOF

(71) Applicant: Washington University, St. Louis, MO (US)

(72) Inventors: Michael Major, St. Louis, MO (US); Brittany Bowman, St. Louis, MO (US); Roland Dolle, St. Louis, MO (US); Matthew Medcalf, St. Louis, MO (US)

(21) Appl. No.: 17/690,826

(22) Filed: Mar. 9, 2022

Related U.S. Application Data
(60) Provisional application No. 63/158,594, filed on Mar. 9, 2021.

(51) Int. Cl. C07D 239/42; C07D 239/49; C07D 491/107; C07D 239/47; C07D 403/10; C07D 413/10; C07D 403/12; A61K 45/06

(52) U.S. Cl. CPC C07D 239/42; C07D 239/49; C07D 491/107; C07D 239/47; C07D 403/10; C07D 413/10; C07D 403/12; A61K 45/06

(57) ABSTRACT

Among the various aspects of the present disclosure is the provision of compositions of NRF2 inhibiting agents and methods of use thereof. These agents can be useful for cancer treatment, including as radiosensitizing agents and as chemotherapeutic sensitizing agents.

### CROSS REFERENCE TO RELATED APPLICATIONS

This application claims the benefit of U.S. Provisional Application No. 63/158,594, filed Mar. 9, 2021, the disclosure of which is hereby incorporated by reference in its entirety.

### FIELD OF THE DISCLOSURE

The present disclosure relates to methods for targeted cancer drug therapies using NRF2 inhibiting agents and to therapeutic compounds for the purpose of targeted therapy of various cancers.

### BACKGROUND

Pyrimethamine (PYR) is a well-studied compound originally used for treating malaria and now is standardly used to treat Toxoplasmosis. PYR is a folate analog which binds to plasmodium and bacterial dihydrofolate reductase (DHFR) protein inhibiting the enzymatic conversion of DHF to THF causing downstream decreases in DNA and protein synthesis. More recently, PYR has been used in clinical trials to treat blood cancers. Historically, however, Methotrexate (MTX), which is another DHFR inhibitor, has been used as an FDA approved anti-folate drug for cancer treatment.

NRF2 is a transcription factor important for the regulation of cellular homeostasis by increasing antioxidant related genes. NRF2 is negatively regulated at the protein level via an E3 ubiquitin ligase complex composed of KEAP1 and CUL3. Cellular stress, including reactive oxygen species, metabolic stress and those caused by chemotherapy and radiation, induces conformational changes in KEAP1 that allow for NRF2 to escape degradation and to drive a gene transcriptional program to restore cell health and proliferation.

NRF2 and KEAP1 are mutated in several types of cancer, resulting in constitutive NRF2-driven transcription and consequently cellular resistance to oxidative and metabolic stress. Beyond mutation, other mechanisms result in NRF2 activation in cancer. Collectively, NRF2 activity drives tumor progression and resistance to chemotherapy and radiation therapy.

Therefore a need exists in the art for pharmacological therapeutic agents which suppress NRF2 activity for cancer treatment, including as agents to sensitize to traditional chemotherapy and radiotherapy.

### SUMMARY

Among the various aspects of the present disclosure is the provision of compositions comprising compounds useful for reducing the expression and/or activity of NRF2 and methods of use thereof.

An aspect of the present disclosure provides for a composition comprising a compound according to Formula (I) or (II) (wherein X can be C, N, S, O, etc.). In some embodiments, R₁-R₈ can be independently selected from nothing (wherein there is no atom, a single bond, or a double bond for correct valence), amino, aminoC₁₋₁₀alkyl (e.g., aminopropyl), bi-cyclo-alkyl-substituted sulfonyl (e.g., bicyclo[3.1.0]hexan-3-ylsulfonyl), bi-hetrocyclyl (e.g., azabicyclo[3.1.0]hexan-3-yl), carboxyl-substituted C₁₋₁₀ alkyl (e.g., N-(3λ3-propyl)pent-4-enamidyl), carboxyl-substituted and heterocyclyl-substituted C₁₋₁₀ alkyl (e.g., 3-(3-(3-(but-3-yn-1-yl)-3H-diazirin-3-yl)propanamido)propanyl), carboxyl-substituted and heterobicyclyl-substituted C₁₋₁₀ alkyl (e.g., 3-(5-((4R)-2-hydroxyhexahydro-1H-thieno[3,4-d]imidazol-4-yl)pentanamido)propanyl), C₁₋₁₀ alkyl (e.g., ethyl, butynyl, pentynyl), C₁₋₁₀ alkoxy-substituted C₁₋₁₀ alkyl (e.g., methoxyethoxyethyl), C₁₋₁₀ alkylsulfonyl (e.g., methylsulfonyl), C₁₋₁₀ cycloalkyl (e.g., cyclobutyl), C₁₋₁₀ heteroclyclyl (e.g., pyrrolidinyl, morpholino), C₃₋₁₀ cycloalkyl-substituted sulfonyl (e.g., cyclopentylsulfonyl), C₃₋₁₀ cycloalkyl-substituted C₁₋₁₀ alkyl (e.g., cyclobutylmethyl), H, halo (e.g., Cl, F), halo-substituted C₁₋₁₀ alkyl (e.g., difluoroethyl, trifluoromethyl), halo-substituted bi-C₃₋₁₀ cycloalkyl (e.g., 3-fluorobicyclo[1.1.1]pentan-1-yl), hetro-bi-cyclo-alkyl-substituted sulfonyl (e.g., azaspiro[3.3]heptan-6-yl)sulfonyl), heterocyclyl-substituted C₁₋₁₀ alkyl, oxo, or oxy-substituted C₁₋₁₀ alkyl (e.g., methoxyethoxyethyl). In some embodiments, R₂ and R₃ are linked (e.g., with C₁₋₁₀ alkyl such as butyl). Another aspect of the present disclosure provides for a composition comprising an NRF2 inhibiting agent according to formula (I) or (II). In some embodiment, X can be C, N, S, O, etc. In some embodiments, R₁ is no atom, H or halo (e.g., Cl); R₂ is H, halo (e.g., Cl), or C₁₋₁₀ cycloalkyl (e.g., cyclobutyl); R₃ is H, halo (e.g., Cl), halo-substituted C₁₋₁₀ alkyl (e.g., difluoroethyl), halo-substituted bi-C₃₋₁₀ cycloalkyl (e.g., 3-fluorobicyclo[1.1.1]pentan-1-yl), C₃₋₁₀ cycloalkyl-substituted sulfonyl (e.g., cyclopentylsulfonyl), hetro-bi-cyclo-alkyl-substituted sulfonyl (e.g., azaspiro[3.3]heptan-6-yl)sulfonyl), bi-cyclo-alkyl-substituted sulfonyl (e.g., bicyclo[3.1.0]hexan-3-ylsulfonyl), or bi-hetrocyclyl (e.g., azabicyclo[3.1.0]hexan-3-yl); R₂ and R₃ are optionally linked with C₁₋₁₀ alkyl (e.g., butyl); R₄ is H, halo (e.g., Cl), halo-substituted C₁₋₁₀ alkyl (e.g., trifluoromethyl), C₁₋₁₀ alkyl sulfonyl (e.g., methylsulfonyl), or C₁₋₁₀ heterocyclyl (e.g., pyrrolidinyl, morpholino); R₅ is H or halo (e.g., F); R₆ is H, aminoC₁₋₁₀alkyl (e.g., aminopropyl), C₁₋₁₀ alkyl (e.g., ethyl, butynyl, pentynyl), C₁₋₁₀ alkoxy-substituted C₁₋₁₀ alkyl (e.g., methoxyethoxyethyl), oxy-substituted C₁₋₁₀ alkyl (e.g., methoxyethoxyethyl), C₃₋₁₀ cycloalkyl-substituted C₁₋₁₀ alkyl (e.g., cyclobutylmethyl), carboxyl-substituted C₁₋₁₀ alkyl (e.g., N-(3λ3-propyl)pent-4-enamidyl), heterocyclyl-substituted C₁₋₁₀ alkyl, carboxyl-substituted and heterocyclyl-substituted C₁₋₁₀ alkyl (e.g., 3-(3-(3-(but-3-yn-1-yl)-3H-diazirin-3-yl)propanamido)propanyl), carboxyl-substituted and heterobicyclyl-substituted C₁₋₁₀ alkyl (e.g., 3-(5-((4R)-2-hydroxyhexahydro-1H-thieno[3,4-d]imidazol-4-yl)pentanamido)propanyl); R₇ is amino or H; or R₈ is amino, H, halo (e.g., Cl), or oxo.

In some embodiments, the compound has an IC₅₀ less than about 1.2 μM. In some embodiments, Formula (I) or (II) is biotin functionalized. In some embodiments, Formula (I) or (II) is not PYR. Yet another aspect of the present disclosure provides for a method of inhibiting or suppressing NRF2 activity or function in a subject. In some embodiments, the method comprises administering an amount of a compound of Formula (I) or (II) according to any one of the preceding aspects or embodiments, in an amount effective to inhibit NRF2 activity or function. In some embodiments, the amount effective to inhibit NRF2 activity or function is an amount that reduces NRF2 protein abundance or accumulation and downstream target gene expression. In some embodiments, the amount effective to inhibit NRF2 activity or function is an amount that results in downstream decreases in DNA and protein synthesis. In some embodiments, the amount effective to inhibit NRF2 activity or function is an amount that decreases NRF2 mRNA, NRF2 protein abundance, or downstream targets. In some embodiments, the amount effective to inhibit NRF2 activity or function is an amount that decreases NRF2 protein abundance. In some embodiments, the amount effective to inhibit NRF2 activity or function is an amount that inhibits NRF2 protein accumulation and activity. In some embodiments, the amount effective to inhibit NRF2 activity or function is an amount that decreases expression of downstream NRF2 target proteins. In some embodiments, the amount effective to inhibit NRF2 activity or function is an amount that decreases NFE2L2, GCLC, GCLM, SLC7a11, or NQO1. In some embodiments, the amount effective to inhibit NRF2 activity or function is an amount that blocks NRF2 activation induced by chemical inhibitors of KEAP1. In some embodiments, the method comprises maintaining proteasomal activity to inhibit NRF2. In some embodiments, the subject has cancer, is suspected of having cancer, or is at risk for having cancer. In some embodiments, the cancer is an NRF2-associated cancer. In some embodiments, the cancer is a cancer with active NRF2 or is an NRF2-activated cancer. In some embodiments, the cancer is associated with NRF2 activation, wherein the NRF2 activation is driven by one or more of an NRF2 mutation, KEAP1 mutation, CUL3 mutation, NRF2 over-expression, or KEAP1 competitive activation. In some embodiments, the cancer is an NRF2-mutated cancer. In some embodiments, the cancer is a KEAP1-mutated cancer. In some embodiments, the cancer is lung, esophageal, kidney, head and neck, ovarian, bladder, or liver cancer. In some embodiments, the cancer is blood cancer or solid tumor. In some embodiments, the method further comprises administrating the NRF2 inhibiting agent in combination with chemotherapy or radiation, separately or together.

Other objects and features will be in part apparent and in part pointed out hereinafter.

### BRIEF DESCRIPTION OF THE DRAWINGS

The application file contains at least one drawing executed in color. Copies of this patent application publication with color drawing(s) will be provided by the Office upon request and payment of the necessary fee.

FIG. 1 shows IC₅₀ calculations for PYR against three NRF2 pathway activators, CDDO-Me 0.1 μM, SULF 2 μM, or PRL295 5 μM in H1299-NQO1-YFP cells. Data was analyzed and graphed using GraphPad Prism. Dotted lines represent confidence intervals of 95% of three independent experiments.

FIG. 2 shows H1299-NQO1-YFP were transfected with WT or mutant KEAP1 to see if PYR still inhibited NRF2 dependent activation from dominant negative KEAP1 mutants. Data is of 3 independent experiments, error bars are SD.

FIG. 3 shows western blot analysis of PYR regulation of NRF2 in KYSE70 cells, esophageal cell line with a NRF2 mutation. KYSE70 cells were treated with increases concentrations of PYR for 48 hours. Representative western blot of two biological experiments.

FIG. 4 shows western blot analysis of PYR regulation of NRF2 in a panel of cell lines with elevated NRF2 treated with PYR (10 μM) or DMSO for 48 hours. Representative western blot of three biological replicates, with quantification of NRF2 protein levels in graph below.

FIG. 5 shows western blot analysis of PYR regulation of NRF2 wild type cell lines. HEK293T cells were co-treated with CDDO (0.1 μM) or Bortezomib (BORT, 0.04 μM) with PYR (10 μM) for 48 hours. H1299 cells were co-treated with BORT (0.04 μM)+/−PYR (10 μM) for 24 hours.

FIG. 6 shows RT-qPCR analysis of KYSE70 cells treated with PYR (10 μM) for 24 or 48 hours. NQO1 and OSGIN1 are NRF2 downstream targets. Error bars are +/−SD and * represents p-value <0.05.

FIG. 7 shows proliferation IC₅₀ calculations for PYR in H1299 or KYSE70 cells. Data was analyzed from the 96-hour time point and graphed using GraphPad Prism. Dotted lines represent confidence intervals of 95% and error bars are SD from three independent experiments.

FIG. 8 shows chemical structures of PYR analogs. Parent compound PYR is boxed. Most changes were to the chloride group, although three compounds, 112, 113, 101 had substitutions or removal of one of the amine groups, respectively. Compound 103 was created without the side ethyl group.

FIG. 9 shows trial doses for first round of PYR analogs. H1299-NQO1-YFP cells were used +/−5 μM PRL295 to activate the NRF2 pathway compared to two doses either 1 or 10 μM of the respective analog.

FIG. 10 shows western blot analysis of HEK293T cells were co-treated with 10 μM of analog 104 or three NRF2 pathway activators for 24 hours. A. PRL295 5 μM. B. CDDO 0.1 μM. C. BORT 0.04 μM.

FIG. 11 shows chemical structures of 104 analogs. Parent compound 104 is boxed. Most changes are to the ring containing the chloride group. Compounds 124 and on tested changes to the length of the original ethyl chain, however 125 removed an amine group, and 134 removed the ethyl chain altogether. Compounds 133 and 133a are based off PYR, having additional methyl groups off the amines to see if bulkiness affects activity.

FIG. 12 shows IC₅₀ calculations for PYR, analogs 104, 114 115 against three NRF2 pathway activators, CDDO-Me 0.1 μM, SULF 2 μM, or PRL295 5 μM in H1299-NQO1-YFP cells. Data was analyzed and graphed using GraphPad Prism. Dotted lines represent confidence intervals of 95% of three independent experiments.

FIG. 13 shows western blot analysis of best analogs in KYSE70, A549, OE21, or PC-9 cells all of which have elevated NRF2. Analog 101 was used as a negative control. Cells were treated for 48 hours with 10 μM PYR, or 1 μM for all analogs. Representative images from 4 independent experiments. NRF2 protein levels are quantified in graphs below, normalized to VINC and then normalized to DMSO.

FIG. 14 shows western blot analysis of H1299 cells were co-treated with 10 μM of PYR or 1 μM of analog 115 and 0.5 μM MLN for 24 hours. Neither PYR nor 115 can decrease NRF2 protein levels with MLN induced stabilization.

FIG. 15 shows RT-qPCR analysis of KYSE70 cells treated with the best PYR analogs (PYR 10 μM, all analogs were treated with 1 μM) for 48 hours. All analogs inhibit NRF2 mRNA and downstream targets. Error bars are +/−SD and * represents p-value <0.05.

FIG. 16 shows proliferation IC₅₀ calculations for analog 115 in H1299 or KYSE70 cells. Data was analyzed from the 96-hour time point and graphed using GraphPad Prism. Dotted lines represent confidence intervals of 95% of one experiment.

FIG. 17A, FIG. 17B, FIG. 17C, FIG. 17D, and FIG. 17E show the mouse study on analog 115. FIG. 17A shows dosing vehicle, administration, pharmacokinetic analysis and clinical observations. FIG. 17B shows plasma concentrations and pharmacokinetic profile. FIG. 17C shows plasma concentrations and pharmacokinetic profile. FIG. 17D shows the standard curve analysis summary in plasma. FIG. 17E shows the bioanalytical acceptance criteria.

FIG. 18 shows chemical structures of 115 analogs. Analogs 139 and 135 are intermediates for 136-138 which were designed for capturing drug protein interactions. Analog 136 has a photo-reactive-diazirine for cross-linking and a “click” chemistry alkyne handle for capture. Analog 137 has a biotin moiety for streptavidin pulldown. Lastly, analog 138 is a negative control for 136 and 137.

FIG. 19 shows IC50 calculations for PYR, and DHFR inhibitors MTX, PEM, and CG against three NRF2 pathway activators, CDDO-Me 0.1 μM, SULF 2 μM, or PRL295 5 μM in H1299-NQO1-YFP cells. Data was analyzed and graphed using GraphPad Prism. Dotted lines represent confidence intervals of 95% of three independent experiments.

FIG. 20 shows western blot analysis of best analogs in KYSE70 and A549, which have elevated NRF2. Cells were treated for 48 hours with 10 μM PYR, 1 μM 115, 0.1 μM MTX, 0.1 μM PEM, and 10 μM CG. Representative images from 3 independent experiments. NRF2 protein levels are quantified in graphs next to, normalized to VINC and then normalized to DMSO.

FIG. 21 shows qRT-PCR of DHFR inhibitors with PYR and 115 as positive controls, show MTX but not PEM suppress NRF2 and NRF2 downstream targets. Both MTX and PEM inhibit AXIN2 and CK1□1 mRNA demonstrating possible off target affects. KYSE70 cells were treated for 48 hours with 10 μM PYR, 1 μM 115, 0.1 μM MTX, and 0.1 μM PEM. Single experiment shown. AXIN2 and CK1γ1 were done in same experiment, but separated for discussion. Error bars are SD and * represents p-value <0.05.

FIG. 22 shows proliferation IC₅₀ calculations for MTX (same as FIGS. 7, 16) compared to analog 115 and PYR in H1299 or KYSE70 cells. Data was analyzed from the 96-hour time point and graphed using GraphPad Prism. Dotted lines represent confidence intervals of 95% of one or two experiments.

FIG. 23 shows western blot analysis of KYSE70 cells co treated with DHFR inhibitors and Folinic Acid (FA) for 48 hours. Concentrations were 10 μM PYR, 1 μM 115, 0.1 μM MTX, 0.1 μM PEM, 10 μM CG and 10 ng/ml of FA. Representative images from 3 independent experiments. NRF2 protein levels are quantified in the graph below, normalized to VINC and then normalized to DMSO without FA.

FIG. 24 shows qRT-PCR of KYSE70 cells treated with DHFR inhibitors and FA for 48 hours. Concentrations were 10 μM PYR, 1 μM 115, 0.1 μM MTX, 0.1 μM PEM, 10 μM CG and 10 ng/ml of FA. Only one replicate, error bars are SD.

FIG. 25 shows H1299 cells were treated for 24 hr with 10 ng/ml FA, 0.1 μM CDDO, 5 μM PRL295, or 0.5 μM MLN compounds. Representative image from 3 biological replicates, NRF2 protein levels quantified in graph on right error bars are SD.

FIG. 26 shows western blot analysis of DHFR CRISPRi KYSE70 cell lines+/−HT withdrawal over 72 hour time point.

FIG. 27 shows a summary of metabolomics study in KYSE70 cells treated with 10 μM PYR, 1 μM 115, and 0.1 μM MTX for 48 hours. Data is of 3 biological replicates normalized to DMSO. Breakdown of data with error included is done for a select number of metabolites in FIGS. 28 and 29.

FIG. 28 shows folate and purine pathways are inhibited. Metabolomics data from 3 biological replicates. Error bars are SD.

FIG. 29 shows methionine and transsulfuration cycles are inhibited. Metabolomics data from 3 biological replicates. Error bars are SD.

### DETAILED DESCRIPTION

Applicant discovered that pyrimethamine (PYR), as well as analogs thereof, selectively kill cancer cells. Specifically, Applicant discovered that PYR at low μM doses can inhibit NRF2 protein accumulation and activity in both NRF2 wild type cells and in NRF2 mutant/active cells. PYR also blocks NRF2 activation induced by chemical inhibitors of KEAP1. Through a series of structure-activity relationship studies, Applicant identified PYR analogs that are ˜20 fold more potent that PYR in NRF2 suppression. Accordingly, provided herein are compositions comprising PYR and analogs of PYR. Primary among the various PYR derivatives is the presence of moving the chlorine group from R3 to R2, having two chlorine groups one at R2 and R4, or substituting the chlorine group for CF3 at position R2, yielded the most robust analogs (Analogs 104, 114, 115) with IC50s of 0.1 and 0.05 respectively. Importantly, several analogs with bulky or long chains, which had IC₅₀s around 0.5 μM (Analogs 126-128) suggesting larger groups at that site would be tolerated and are amenable to the addition of, for example, biotin or photo-reactive-diazirine for cross-linking and a “click” chemistry alkyne handle for capture. Indeed, Analog 136 has a photo-reactive-diazirne and has an attractive IC₅₀ of around 0.15 μM. Also provided herein are methods of using PYR or analogs of PYR to inhibit the growth, proliferation, and metastasis of cancer cells. PYR or analogs of PYR, therefore, may be used to treat a cancer or tumor.

Discussed below are components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### I. Compositions

In an aspect, a composition of the disclosure comprises pyrimethamine (PYR) or a pyrimethamine analog. A PYR or a PYR analog as disclosed herein may be modified to improve potency, bioavailability, solubility, stability, handling properties, or a combination thereof, as compared to an unmodified version. Thus, in another aspect, a composition of the disclosure comprises a modified PYR or PYR analog. In still another aspect, a composition of the disclosure comprises a prodrug of a PYR or PYR analog.

A composition of the disclosure may optionally further comprise one or more PYR or PYR analog and/or one or more additional drug or therapeutically active agent. A composition of the disclosure may further comprise a pharmaceutically acceptable excipient, carrier or diluent. Further, a composition of the disclosure may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents or antioxidants.

Other aspects of the disclosure are described in further detail below.

### (a) Pyrimethamine (PYR) and Pyrimethamine Analogs

In general, the compounds detailed herein include compounds comprising a pyrimethamine structure as diagrammed below. Pyrimethamine (Daraprim) is a small molecule developed in the 1950s and is currently used to treat parasitic diseases and malaria. Pyrimethamine is being explored for slowing Tay-Sachs and for treating ALS. Pyrimethamine is a synthetic derivative of ethyl-pyrimidine with potent antimalarial properties. Synthesis of pyrimethamine typically begins with p-chlorophenylacetonitrile, which undergoes a condensation reaction with ethyl propionate ester; the product of this then reacts with diazomethane to form an enol ether, which reacts with free guanidine in a second condensation reaction. Thus, PYR and analogs thereof can be produced by organic synthesis.

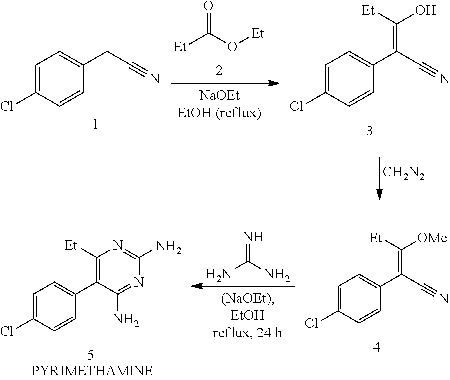

The present disclosure is based, at least in part, on the discovery that Pyrimethamine and/or PYR analogs are useful as a potent inhibitor of NRF2 activity. As shown herein, analogs of Pyrimethamine were created that suppress or inhibit NRF2 activity by ˜20-fold compared to Pyrimethamine. These small molecules can be further developed as cancer therapeutics or to treat other conditions associated with NRF2 overexpression including as agents to sensitize to traditional chemotherapy and radiotherapy.

NRF2 is a transcription factor that regulates the expression of multiple genes key in protecting against oxidative damage. NRF2 has been proposed as a therapeutic target for neurodegenerative, respiratory, cardiovascular, and cancer indications. Constitutive activation of NRF2 promotes the development of many cancer types and increases cancer resistance to radiation and chemotherapy. Reducing NRF2 activity reverses drug and radiation sensitivity. NRF2 inhibitors have been proposed as potential cancer therapeutics, but none have been FDA approved. Pyrimethamine has an excellent safety profile.

Thus provided herein are analogs of PYR. PYR derivatives are modified versions of PYR that are able to inhibit NRF2 expression and/or activity. As used herein a “PYR analog” may be a PYR analog known in the art or a PYR analog of Formula (I) or (II).

As such, provided herein are compounds comprising Formula (I):

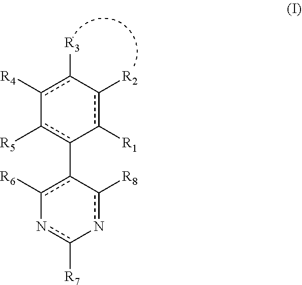

wherein:
R₁, R₂, R₃, R₄, R₅, R₆, R₇, and R₈ are independently selected from the group consisting of no atom, amino, amino-C₁₋₁₀ alkyl (e.g., aminopropyl), bi-cyclo-alkyl-substituted sulfonyl (e.g., bicyclo[3.1.0]hexan-3-ylsulfonyl), bi-hetrocyclyl (e.g., azabicyclo[3.1.0]hexan-3-yl), carboxyl-substituted-C₁₋₁₀ alkyl (e.g., N-(3λ3-propyl)pent-4-enamidyl), carboxyl-substituted- or heterocyclyl-substituted-C₁₋₁₀ alkyl (e.g., 3-(3-(3-(but-3-yn-1-yl)-3H-diazirin-3-yl)propanamido)propanyl), carboxyl-substituted- or heterobicyclyl-substituted-C₁₋₁₀ alkyl (e.g., 3-(5-((4R)-2-hydroxyhexahydro-1H-thieno[3,4-d]imidazol-4-yl)pentanamido)propanyl), C₁₋₁₀ alkyl (e.g., ethyl, butynyl, pentynyl), C₁₋₁₀ alkoxy-substituted C₁₋₁₀ alkyl (e.g., methoxyethoxyethyl), C₁₋₁₀ alkylsulfonyl (e.g., methylsulfonyl), C₁₋₁₀ cycloalkyl (e.g., cyclobutyl), C₁₋₁₀ heteroclyclyl (e.g., pyrrolidinyl, morpholino), C₃₋₁₀ cycloalkyl-substituted sulfonyl (e.g., cyclopentylsulfonyl), C₃₋₁₀ cycloalkyl-substituted C₁₋₁₀ alkyl (e.g., cyclobutylmethyl), H, halo (e.g., Cl, F), halo-substituted C₁₋₁₀ alkyl (e.g., difluoroethyl, trifluoromethyl), halo-substituted bi-C₃₋₁₀ cycloalkyl (e.g., 3-fluorobicyclo[1.1.1]pentan-1-yl), hetro-bi-cyclo-alkyl-substituted sulfonyl (e.g., azaspiro[3.3]heptan-6-yl)sulfonyl), heterocyclyl-substituted C₁₋₁₀ alkyl, oxo- or oxy-substituted C₁₋₁₀ alkyl (e.g., methoxyethoxyethyl), and optionally, R2 and R3 are linked for example with C₁₋₁₀ alkyl, such as butyl.

In an embodiment, a compound of Formula (I) comprises any of the preceding compounds of Formula (I), wherein R₁ may be selected from the group consisting of no atom, H or halo. In a particular embodiment, a compound of Formula (I) comprises any of the proceeding compounds of Formula (I), wherein R₁ is H. In another particular embodiment, a compound of Formula (I) comprises any of the proceeding compounds of Formula (I), wherein R₁ is Cl.

In another embodiment, a compound of Formula (I) comprises any of the preceding compounds of Formula (I), wherein R₂ is selected from the group consisting of H, halo, C₁₋₁₀ cycloalkyl and where R₂ and R₃ are linked with C₁₋₁₀ alkyl. In a particular embodiment, a compound of Formula (I) comprises any of the proceeding compounds of Formula (I), wherein R₂ is H. In another particular embodiment, a compound of Formula (I) comprises any of the proceeding compounds of Formula (I), wherein R₂ is cyclobutyl. In still another particular embodiment, a compound of Formula (I) comprises any of the proceeding compounds of Formula (I), wherein R₂ and R₃ are linked with butyl.

In another embodiment, a compound of Formula (I) comprises any of the preceding compounds of Formula (I) where R₂ and R₃ are not linked, wherein R₃ is selected from the group consisting of H, halo, halo-substituted C₁₋₁₀ alkyl, C₃₋₁₀ cycloalkyl-substituted sulfonyl, halo-substituted bi-C₃₋₁₀ cycloalkyl, hetro-bi-cyclo-alkyl-substituted sulfonyl or bi-cyclo-alkyl-substituted sulfonyl, and bi-hetrocyclyl. In a particular embodiment, a compound of Formula (I) comprises any of the proceeding compounds of Formula (I) where R₂ and R₃ are not linked, wherein R₃ is H. In another particular embodiment, a compound of Formula (I) comprises any of the proceeding compounds of Formula (I) where R₂ and R₃ are not linked, wherein R₃ is Cl. In still another particular embodiment, a compound of Formula (I) comprises any of the proceeding compounds of Formula (I) where R₂ and R₃ are not linked, wherein R₃ is cyclopentylsulfonyl. In yet another particular embodiment, a compound of Formula (I) comprises any of the proceeding compounds of Formula (I) where R₂ and R₃ are not linked, wherein R₃ is azaspiro[3.3]heptan-6-yl)sulfonyl. In still yet another particular embodiment, a compound of Formula (I) comprises any of the proceeding compounds of Formula (I) where R₂ and R₃ are not linked, wherein R₃ is bicyclo[3.1.0]hexan-3-ylsulfonyl. In still another particular embodiment, a compound of Formula (I) comprises any of the proceeding compounds of Formula (I) where R₂ and R₃ are not linked, wherein R₃ is 3-fluorobicyclo[1.1.1]pentan-1-yl. In yet another particular embodiment, a compound of Formula (I) comprises any of the proceeding compounds of Formula (I) where R₂ and R₃ are not linked, wherein R₃ is azabicyclo[3.1.0]hexan-3-yl.

In another embodiment, a compound of Formula (I) comprises any of the preceding compounds of Formula (I), wherein R₄ is selected from the group consisting of H, halo, halo-substituted C₁₋₁₀ alkyl, C₁₋₁₀ alkylsulfonyl, and C₁₋₁₀ heterocyclyl. In a particular embodiment, a compound of Formula (I) comprises any of the proceeding compounds of Formula (I), wherein R₄ is H. In another particular embodiment, a compound of Formula (I) comprises any of the proceeding compounds of Formula (I), wherein R₄ is Cl. In still another particular embodiment, a compound of Formula (I) comprises any of the proceeding compounds of Formula (I), wherein R₄ is trifluoromethyl. In yet another particular embodiment, a compound of Formula (I) comprises any of the proceeding compounds of Formula (I), wherein R₄ is methylsulfonyl. In still yet another particular embodiment, a compound of Formula (I) comprises any of the proceeding compounds of Formula (I), wherein R₄ is pyrrolidin-1-yl. In still another particular embodiment, a compound of Formula (I) comprises any of the proceeding compounds of Formula (I), wherein R₄ is morpholino.

In another embodiment, a compound of Formula (I) comprises any of the preceding compounds of Formula (I), wherein R₅ is selected from the group consisting of H and halo. In a particular embodiment, a compound of Formula (I) comprises any of the proceeding compounds of Formula (I), wherein R₅ is H. In another particular embodiment, a compound of Formula (I) comprises any of the proceeding compounds of Formula (I), wherein R₅ is F.

In another embodiment, a compound of Formula (I) comprises any of the preceding compounds of Formula (I), wherein R₆ is selected from the group consisting of H, aminoC₁₋₁₀alkyl, C₁₋₁₀ alkyl, C₁₋₁₀ alkoxy-substituted C₁₋₁₀ alkyl, oxy-substituted C₁₋₁₀ alkyl, C₃₋₁₀ cycloalkyl-substituted C₁₋₁₀ alkyl, carboxyl-substituted C₁₋₁₀ alkyl, heterocyclyl-substituted C₁₋₁₀ alkyl, carboxyl-substituted- or heterocyclyl-substituted-C₁₋₁₀ alkyl, and carboxyl-substituted- or heterobicyclyl-substituted-C₁₋₁₀ alkyl. In a particular embodiment, a compound of Formula (I) comprises any of the proceeding compounds of Formula (I), wherein R₆ is H. In another particular embodiment, a compound of Formula (I) comprises any of the proceeding compounds of Formula (I), wherein R₆ is ethyl. In still another particular embodiment, a compound of Formula (I) comprises any of the proceeding compounds of Formula (I), wherein R₆ is but-3-yn-1-yl. In yet another particular embodiment, a compound of Formula (I) comprises any of the proceeding compounds of Formula (I), wherein R₆ is methoxyethoxy ethyl. In still yet another particular embodiment, a compound of Formula (I) comprises any of the proceeding compounds of Formula (I), wherein R₆ is cyclobutylmethyl. In still another particular embodiment, a compound of Formula (I) comprises any of the proceeding compounds of Formula (I), wherein R₆ is pent-4-yn-1-yl. In still another particular embodiment, a compound of Formula (I) comprises any of the proceeding compounds of Formula (I), wherein R₆ is pentyl. In still yet another particular embodiment, a compound of Formula (I) comprises any of the proceeding compounds of Formula (I), wherein R₆ is but-3-en-1-yl. In yet another particular embodiment, a compound of Formula (I) comprises any of the proceeding compounds of Formula (I), wherein R₆ is aminopropyl. In another particular embodiment, a compound of Formula (I) comprises any of the proceeding compounds of Formula (I), wherein R₆ is 3-(3-(3-(but-3-yn-1-yl)-3H-diazirin-3-yl)propanamido)propanyl. In yet another particular embodiment, a compound of Formula (I) comprises any of the proceeding compounds of Formula (I), wherein R₆ is 3-(5-((4R)-2-hydroxyhexahydro-1H-thieno[3,4-d]imidazol-4-yl)pentanamido)propanyl. In yet another particular embodiment, a compound of Formula (I) comprises any of the proceeding compounds of Formula (I), wherein R₆ is N-(3λ3-propyl)pent-4-enamidyl.

In another embodiment, a compound of Formula (I) comprises any of the preceding compounds of Formula (I), wherein R₇ is selected from the group consisting of amino and H. In a particular embodiment, a compound of Formula (I) comprises any of the proceeding compounds of Formula (I), wherein R₇ is H. In another particular embodiment, a compound of Formula (I) comprises any of the proceeding compounds of Formula (I), wherein R₇ is amino. In still another particular embodiment, a compound of Formula (I) comprises any of the proceeding compounds of Formula (I), wherein R₇ is methyl amino.

In another embodiment, a compound of Formula (I) comprises any of the preceding compounds of Formula (I), wherein R₈ is selected from the group consisting of amino, H, halo, and oxo. In a particular embodiment, a compound of Formula (I) comprises any of the proceeding compounds of Formula (I), wherein R₈ is H. In another particular embodiment, a compound of Formula (I) comprises any of the proceeding compounds of Formula (I), wherein R₈ is amino. In still another particular embodiment, a compound of Formula (I) comprises any of the proceeding compounds of Formula (I), wherein R₈ is oxo. In still yet another particular embodiment, a compound of Formula (I) comprises any of the proceeding compounds of Formula (I), wherein R₈ is Cl. In yet another particular embodiment, a compound of Formula (I) comprises any of the proceeding compounds of Formula (I), wherein R₈ is methyl amino.

In certain embodiments a compound of Formula (I), R₂ and R₄, R₁ and R₅, and R₆ and R₈ can be interchangeable.

Also provided herein are compounds comprising Formula (II):

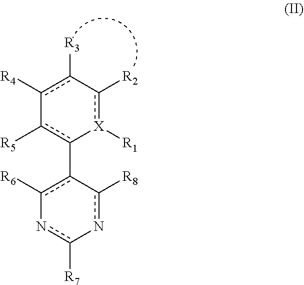

wherein:
X is C, N, S, or O; and
R₁, R₂, R₃, R₄, R₅, R₆, R₇, and R₈ are independently selected from the group consisting of no atom, amino, amino-C₁₋₁₀ alkyl (e.g., aminopropyl), bi-cyclo-alkyl-substituted sulfonyl (e.g., bicyclo[3.1.0]hexan-3-ylsulfonyl), bi-hetrocyclyl (e.g., azabicyclo[3.1.0]hexan-3-yl), carboxyl-substituted-C₁₋₁₀ alkyl (e.g., N-(3λ3-propyl)pent-4-enamidyl), carboxyl-substituted- or heterocyclyl-substituted-C₁₋₁₀ alkyl (e.g., 3-(3-(3-(but-3-yn-1-yl)-3H-diazirin-3-yl)propanamido)propanyl), carboxyl-substituted- or heterobicyclyl-substituted-C₁₋₁₀ alkyl (e.g., 3-(5-((4R)-2-hydroxyhexahydro-1H-thieno[3,4-d]imidazol-4-yl)pentanamido)propanyl), C₁₋₁₀ alkyl (e.g., ethyl, butynyl, pentynyl), C₁₋₁₀ alkoxy-substituted C₁₋₁₀ alkyl (e.g., methoxyethoxyethyl), C₁₋₁₀ alkylsulfonyl (e.g., methylsulfonyl), C₁₋₁₀ cycloalkyl (e.g., cyclobutyl), C₁₋₁₀ heteroclyclyl (e.g., pyrrolidinyl, morpholino), C₃₋₁₀ cycloalkyl-substituted sulfonyl (e.g., cyclopentylsulfonyl), C₃₋₁₀ cycloalkyl-substituted C₁₋₁₀ alkyl (e.g., cyclobutylmethyl), H, halo (e.g., Cl, F), halo-substituted C₁₋₁₀ alkyl (e.g., difluoroethyl, trifluoromethyl), halo-substituted bi-C₃₋₁₀ cycloalkyl (e.g., 3-fluorobicyclo[1.1.1]pentan-1-yl), hetro-bi-cyclo-alkyl-substituted sulfonyl (e.g., azaspiro[3.3]heptan-6-yl)sulfonyl), heterocyclyl-substituted C₁₋₁₀ alkyl, oxo- or oxy-substituted C₁₋₁₀ alkyl (e.g., methoxyethoxyethyl), and optionally, R2 and R3 are linked for example with C₁₋₁₀ alkyl, such as butyl.

In an embodiment, a compound of Formula (II) comprises any of the preceding compounds of Formula (II), wherein X may be selected from the group consisting of C and N. In a particular embodiment, a compound of Formula (II) comprises any of the proceeding compounds of Formula (II), wherein X is C. In another particular embodiment, a compound of Formula (II) comprises any of the proceeding compounds of Formula (II), wherein X is N and R₁ is no atom.

In an embodiment, a compound of Formula (II) comprises any of the preceding compounds of Formula (II), wherein R₁ may be selected from the group consisting of no atom, H or halo. In a particular embodiment, a compound of Formula (I) comprises any of the proceeding compounds of Formula (II), wherein R₁ is H. In another particular embodiment, a compound of Formula (II) comprises any of the proceeding compounds of Formula (II), wherein R₁ is Cl.

In another embodiment, a compound of Formula (II) comprises any of the preceding compounds of Formula (II), wherein R₂ is selected from the group consisting of H, halo, C₁₋₁₀ cycloalkyl and where R₂ and R₃ are linked with C₁₋₁₀ alkyl. In a particular embodiment, a compound of Formula (II) comprises any of the proceeding compounds of Formula (II), wherein R₂ is H. In another particular embodiment, a compound of Formula (II) comprises any of the proceeding compounds of Formula (II), wherein R₂ is cyclobutyl. In still another particular embodiment, a compound of Formula (II) comprises any of the proceeding compounds of Formula (II), wherein R₂ and R₃ are linked with butyl.

In another embodiment, a compound of Formula (II) comprises any of the preceding compounds of Formula (II) where R₂ and R₃ are not linked, wherein R₃ is selected from the group consisting of H, halo, halo-substituted C₁₋₁₀ alkyl, C₃₋₁₀ cycloalkyl-substituted sulfonyl, halo-substituted bi-C₃₋₁₀ cycloalkyl, hetro-bi-cyclo-alkyl-substituted sulfonyl or bi-cyclo-alkyl-substituted sulfonyl, and bi-hetrocyclyl. In a particular embodiment, a compound of Formula (II) comprises any of the proceeding compounds of Formula (II) where R₂ and R₃ are not linked, wherein R₃ is H. In another particular embodiment, a compound of Formula (II) comprises any of the proceeding compounds of Formula (II) where R₂ and R₃ are not linked, wherein R₃ is Cl. In still another particular embodiment, a compound of Formula (II) comprises any of the proceeding compounds of Formula (II) where R₂ and R₃ are not linked, wherein R₃ is cyclopentylsulfonyl. In yet another particular embodiment, a compound of Formula (II) comprises any of the proceeding compounds of Formula (II) where R₂ and R₃ are not linked, wherein R₃ is azaspiro[3.3]heptan-6-yl)sulfonyl. In still yet another particular embodiment, a compound of Formula (II) comprises any of the proceeding compounds of Formula (II) where R₂ and R₃ are not linked, wherein R₃ is bicyclo[3.1.0]hexan-3-ylsulfonyl. In still another particular embodiment, a compound of Formula (II) comprises any of the proceeding compounds of Formula (II) where R₂ and R₃ are not linked, wherein R₃ is 3-fluorobicyclo[1.1.1]pentan-1-yl. In yet another particular embodiment, a compound of Formula (II) comprises any of the proceeding compounds of Formula (II) where R₂ and R₃ are not linked, wherein R₃ is azabicyclo[3.1.0]hexan-3-yl.

In another embodiment, a compound of Formula (II) comprises any of the preceding compounds of Formula (II), wherein R₄ is selected from the group consisting of H, halo, halo-substituted C₁₋₁₀ alkyl, C₁₋₁₀ alkylsulfonyl, and C₁₋₁₀ heterocyclyl. In a particular embodiment, a compound of Formula (II) comprises any of the proceeding compounds of Formula (II), wherein R₄ is H. In another particular embodiment, a compound of Formula (II) comprises any of the proceeding compounds of Formula (II), wherein R₄ is Cl. In still another particular embodiment, a compound of Formula (II) comprises any of the proceeding compounds of Formula (II), wherein R₄ is trifluoromethyl. In yet another particular embodiment, a compound of Formula (II) comprises any of the proceeding compounds of Formula (II), wherein R₄ is methylsulfonyl. In still yet another particular embodiment, a compound of Formula (II) comprises any of the proceeding compounds of Formula (II), wherein R₄ is pyrrolidin-1-yl. In still another particular embodiment, a compound of Formula (II) comprises any of the proceeding compounds of Formula (II), wherein R₄ is morpholino.

In another embodiment, a compound of Formula (II) comprises any of the preceding compounds of Formula (II), wherein R₅ is selected from the group consisting of H and halo. In a particular embodiment, a compound of Formula (II) comprises any of the proceeding compounds of Formula (II), wherein R₅ is H. In another particular embodiment, a compound of Formula (II) comprises any of the proceeding compounds of Formula (II), wherein R₅ is F.

In another embodiment, a compound of Formula (II) comprises any of the preceding compounds of Formula (II), wherein R₆ is selected from the group consisting of H, aminoC₁₋₁₀alkyl, C₁₋₁₀ alkyl, C₁₋₁₀ alkoxy-substituted C₁₋₁₀ alkyl, oxy-substituted C₁₋₁₀ alkyl, C₃₋₁₀ cycloalkyl-substituted C₁₋₁₀ alkyl, carboxyl-substituted C₁₋₁₀ alkyl, heterocyclyl-substituted C₁₋₁₀ alkyl, carboxyl-substituted- or heterocyclyl-substituted-C₁₋₁₀ alkyl, and carboxyl-substituted- or heterobicyclyl-substituted-C₁₋₁₀ alkyl. In a particular embodiment, a compound of Formula (II) comprises any of the proceeding compounds of Formula (II), wherein R₆ is H. In another particular embodiment, a compound of Formula (II) comprises any of the proceeding compounds of Formula (II), wherein R₆ is ethyl. In still another particular embodiment, a compound of Formula (II) comprises any of the proceeding compounds of Formula (II), wherein R₆ is but-3-yn-1-yl. In yet another particular embodiment, a compound of Formula (II) comprises any of the proceeding compounds of Formula (II), wherein R₆ is methoxyethoxy ethyl. In still yet another particular embodiment, a compound of Formula (II) comprises any of the proceeding compounds of Formula (II), wherein R₆ is cyclobutylmethyl. In still another particular embodiment, a compound of Formula (II) comprises any of the proceeding compounds of Formula (II), wherein R₆ is pent-4-yn-1-yl. In still another particular embodiment, a compound of Formula (II) comprises any of the proceeding compounds of Formula (II), wherein R₆ is pentyl. In still yet another particular embodiment, a compound of Formula (II) comprises any of the proceeding compounds of Formula (II), wherein R₆ is but-3-en-1-yl. In yet another particular embodiment, a compound of Formula (II) comprises any of the proceeding compounds of Formula (II), wherein R₆ is aminopropyl. In another particular embodiment, a compound of Formula (II) comprises any of the proceeding compounds of Formula (II), wherein R₆ is 3-(3-(3-(but-3-yn-1-yl)-3H-diazirin-3-yl)propanamido)propanyl. In yet another particular embodiment, a compound of Formula (II) comprises any of the proceeding compounds of Formula (II), wherein R₆ is 3-(5-((4R)-2-hydroxyhexahydro-1H-thieno[3,4-d]imidazol-4-yl)pentanamido)propanyl. In yet another particular embodiment, a compound of Formula (II) comprises any of the proceeding compounds of Formula (II), wherein R₆ is N-(3λ3-propyl)pent-4-enamidyl.

In another embodiment, a compound of Formula (II) comprises any of the preceding compounds of Formula (II), wherein R₇ is selected from the group consisting of amino and H. In a particular embodiment, a compound of Formula (II) comprises any of the proceeding compounds of Formula (II), wherein R₇ is H. In another particular embodiment, a compound of Formula (II) comprises any of the proceeding compounds of Formula (II), wherein R₇ is amino. In still another particular embodiment, a compound of Formula (II) comprises any of the proceeding compounds of Formula (II), wherein R₇ is methyl amino.

In another embodiment, a compound of Formula (II) comprises any of the preceding compounds of Formula (II), wherein R₈ is selected from the group consisting of amino, H, halo, and oxo. In a particular embodiment, a compound of Formula (II) comprises any of the proceeding compounds of Formula (II), wherein R₈ is H. In another particular embodiment, a compound of Formula (II) comprises any of the proceeding compounds of Formula (II), wherein R₈ is amino. In still another particular embodiment, a compound of Formula (II) comprises any of the proceeding compounds of Formula (II), wherein R₈ is oxo. In still yet another particular embodiment, a compound of Formula (II) comprises any of the proceeding compounds of Formula (II), wherein R₈ is Cl. In yet another particular embodiment, a compound of Formula (II) comprises any of the proceeding compounds of Formula (II), wherein R₈ is methyl amino.

In certain embodiments a compound of Formula (II), R₂ and R₄, R₁ and R₅, and R₆ and R₈ can be interchangeable.

Compounds of the above formulas and R groups thereof can be optionally substituted or functionalized (e.g., addition or substitution of a functional group or chemical moiety) with one or more groups independently selected from the group consisting of hydroxyl; C₁₋₁₀ alkyl hydroxyl; amine; C₁₋₁₀ carboxylic acid; C₁₋₁₀ carboxyl; straight chain or branched C₁₋₁₀ alkyl, optionally containing unsaturation; a C₂₋₁₀cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted or functionalized with one or more groups independently selected from the group consisting of hydroxyl; C₁₋₁₀ alkyl hydroxyl; amine; C₁₋₁₀ carboxyl; C₁₋₁₀ carboxylic acid; C₁₋₁₀ carboxyl; straight chain or branched C₁₋₁₀ alkyl, optionally containing unsaturation; straight chain or branched C₁₋₁₀ alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀ alkyl hydroxyl; amine; C₁₋₁₀ carboxylic acid; C₁₋₁₀carboxyl; straight chain or branched C₁₋₁₀ alkyl, optionally containing unsaturation; straight chain or branched C₁₋₁₀ alkyl amine, optionally containing unsaturation; a C₂₋₁₀cycloalkyl optionally containing unsaturation or one oxygen or nitrogen atom; heterocyclyl; straight chain or branched C₁₋₁₀ alkyl amine; heterocyclic amine; and aryl comprising a phenyl; and heteroaryl containing from 1 to 4 N, O, or S atoms. Any of the above can be further optionally substituted or functionalized.

In some embodiments, a compound of Formula (I) or Formula (II) is not pyrimethamine or 5-(3-chlorophenyl)-6-ethylpyrimidine-2,4-diamine (WCDD104).

In one embodiment a compound of Formula (I) is selected from the group consisting of

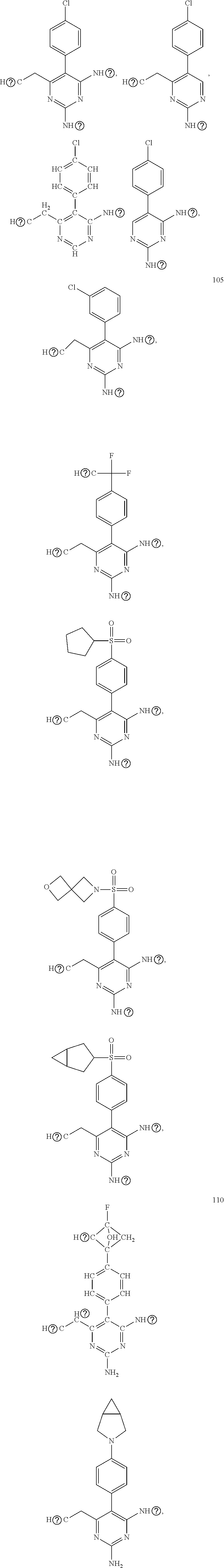

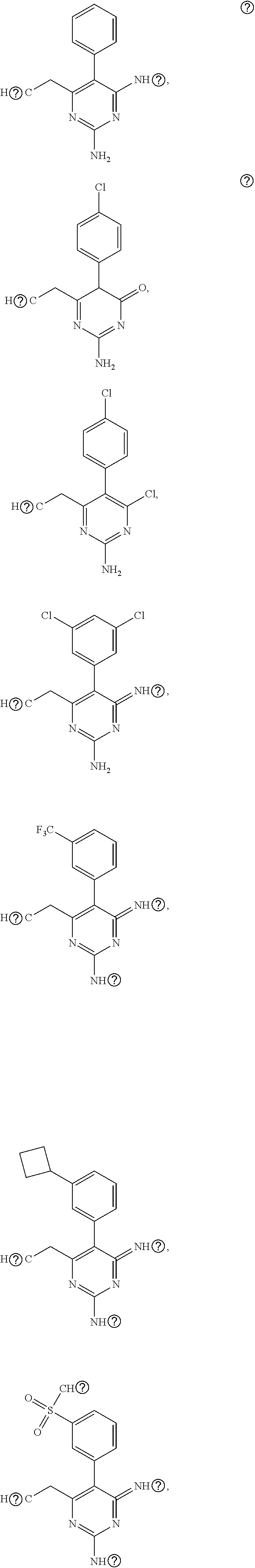

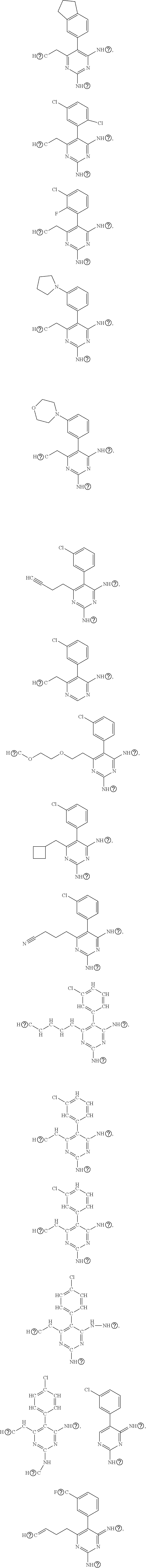

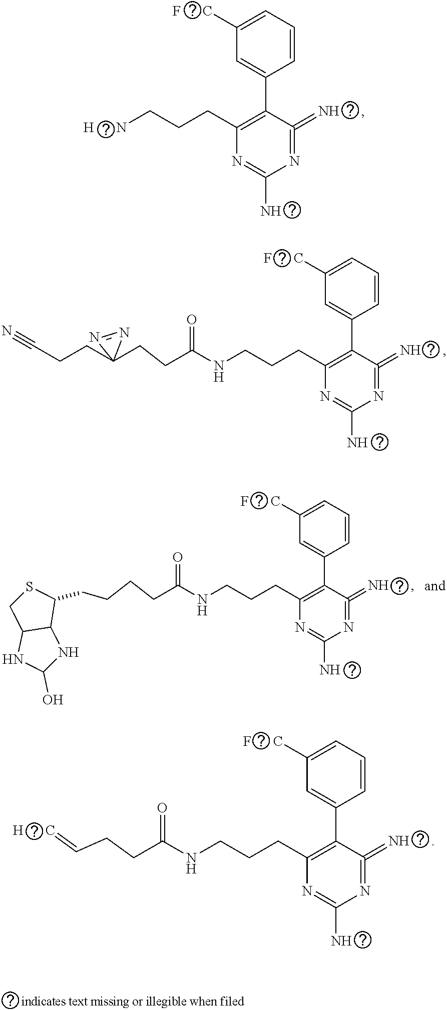

In one embodiment a compound of Formula (II) is selected from the group consisting of

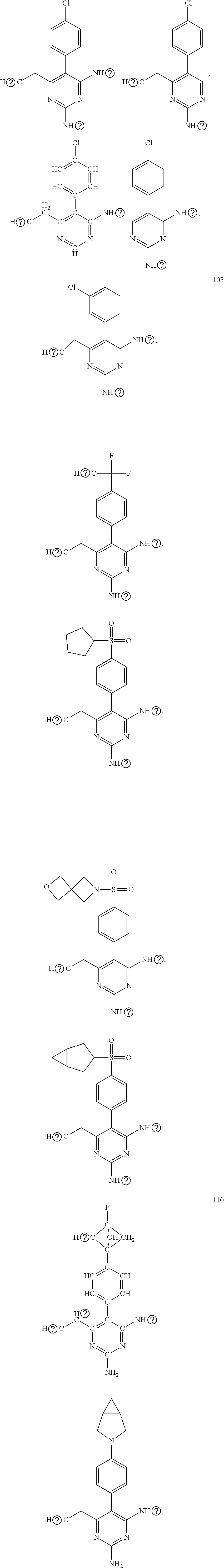

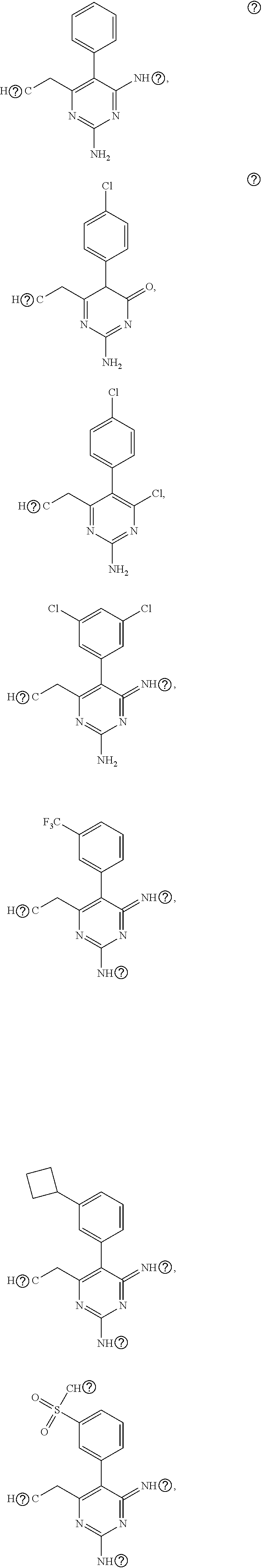

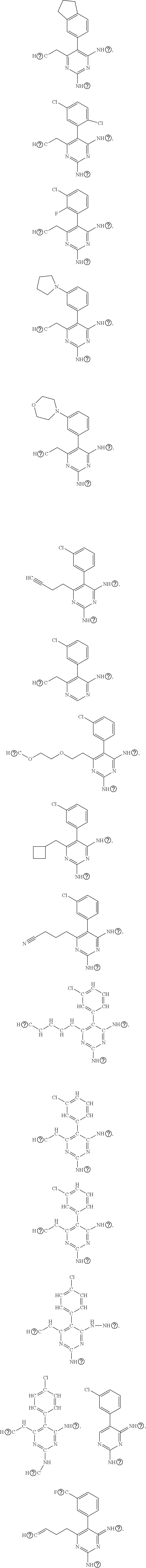

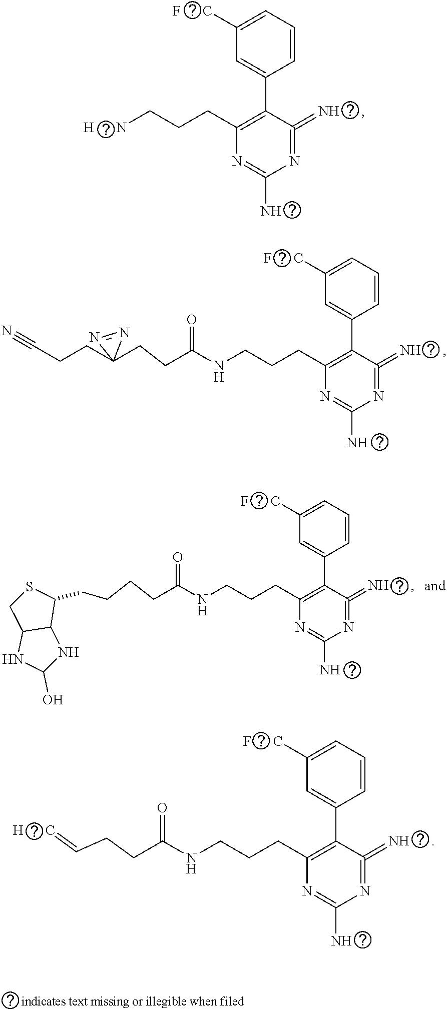

### (b) Components of the Composition

The present disclosure also provides pharmaceutical compositions. The pharmaceutical composition comprises PYR or a PYR analog as disclosed herein, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising SL or a SL derivative is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present disclosure. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the PYR or a PYR analog as disclosed herein in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the PYR or a PYR analog as disclosed herein may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which sphingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the SL or SL derivative (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the disclosure may be delivered to a cell as a microemulsion, nanoemulsion or self-emulsifying system. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The SL or SL derivative may be encapsulated in a microemulsion by any method generally known in the art. Nanoemulsions have a 20 to 500 nm size range and are kinetically stable, and self-emulsifying systems form spontaneously without agitation.

In yet another embodiment, a PYR or a PYR analog as disclosed herein may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Dosages of a compound as disclosed herein can vary between wide limits, depending upon the disease or disorder to be treated and/or the age and condition of the subject to be treated. In an embodiment where a composition comprising a compound of the disclosure is contacted with a sample, the concentration of the compound may be from about 1 μM to about 40 μM. Alternatively, the concentration of the compound of the disclosure may be from about 5 μM to about 25 μM. For example, the concentration of the compound of the disclosure may be about 1, about 2.5 about 5, about 6, about 7, about 8, about 9, about 10, about 11, about 12, about 12, about 14, about 15, about 16, about 17, about 18, about 19, about 20, about 21, about 22, about 23, about 24, about 25, about 30, about 35, or about 40 μM. Additionally, the concentration of the compound of the disclosure may be greater than 40 μM. For example, the concentration of the compound of the disclosure may be about 40, about 45, about 50, about 55, about 60, about 65, about 70, about 75, about 80, about 85, about 90, about 95 or about 100 μM. In certain embodiments, the concentration of the compound of the disclosure may be from about 1 μM to about 10 μM, from about 10 μM to about 20 μM, from about 20 μM to about 30 μM, or from about 30 μM to about 40 μM. In a specific embodiment, the concentration of the compound of the disclosure may be from about 1 μM to about 10 μM.

In an embodiment where the composition comprising a compound of the disclosure is administered to a subject, the dose of the compound of the disclosure may be from about 0.1 mg/kg to about 500 mg/kg. For example, the dose of the compound of the disclosure may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the compound of the disclosure may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the compound of the disclosure may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

The quantity of a pharmaceutical composition necessary to deliver a therapeutically effective dose can be determined by routine in vitro and in vivo methods, common in the art of drug testing. See, for example, D. B. Budman, A. H. Calvert, E. K. Rowinsky (editors). Handbook of Anticancer Drug Development, L W W, 2003. Therapeutically effective dosages for various therapeutic entities are well known to those of skill in the art.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of a composition of the disclosure can occur as a single event or over a time course of treatment. For example, one or more of a nanoparticle composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a cancer or tumor. A compositions of the disclosure can be administered simultaneously or sequentially with another agent, such as an antibiotic, an anti-inflammatory, or another agent. For example, compositions as disclosed herein can be administered simultaneously with another agent, such as a chemotherapeutic, radiation, an antibiotic, or an anti-inflammatory. Simultaneous administration can occur through administration of separate compositions, each containing one or more active agents. Simultaneous administration can occur through administration of one composition containing two or more active agents. A composition as disclosed herein can be administered sequentially with a chemotherapeutic agent, radiation, an antibiotic, an anti-inflammatory, or another agent. For example, a composition of the disclosure can be administered before or after administration of a chemotherapeutic, radiation, an antibiotic, an anti-inflammatory, or another agent.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the disclosure, the subject may be a human or any other animal.

### II. Methods

A further aspect of the present disclosure provides a method for inhibiting growth of a cancer cell. Yet another aspect of the present disclosure provides for a method of inhibiting or suppressing NRF2 activity or function in a subject. In some embodiments, the method comprises administering an amount of a compound of the disclosure, in an amount effective to inhibit NRF2 activity or function. In some embodiments, the amount effective to inhibit NRF2 activity or function is an amount that reduces NRF2 protein abundance or accumulation and downstream target gene expression. In some embodiments, the amount effective to inhibit NRF2 activity or function is an amount that results in downstream decreases in DNA and protein synthesis. In some embodiments, the amount effective to inhibit NRF2 activity or function is an amount that decreases NRF2 mRNA, NRF2 protein abundance, or downstream targets. In some embodiments, the amount effective to inhibit NRF2 activity or function is an amount that decreases NRF2 protein abundance. In some embodiments, the amount effective to inhibit NRF2 activity or function is an amount that inhibits NRF2 protein accumulation and activity. In some embodiments, the amount effective to inhibit NRF2 activity or function is an amount that decreases expression of downstream NRF2 target proteins. In some embodiments, the amount effective to inhibit NRF2 activity or function is an amount that decreases NFE2L2, GCLC, GCLM, SLC7a11, or NQO1. In some embodiments, the amount effective to inhibit NRF2 activity or function is an amount that blocks NRF2 activation induced by chemical inhibitors of KEAP1. In some embodiments, the method comprises maintaining proteasomal activity to inhibit NRF2. In some embodiments, the subject has cancer, is suspected of having cancer, or is at risk for having cancer. In some embodiments, the cancer is an NRF2-associated cancer. In some embodiments, the cancer is a cancer with active NRF2 or is an NRF2-activated cancer. In some embodiments, the cancer is associated with NRF2 activation, wherein the NRF2 activation is driven by one or more of an NRF2 mutation, KEAP1 mutation, CUL3 mutation, NRF2 over-expression, or KEAP1 competitive activation. In some embodiments, the cancer is an NRF2-mutated cancer. In some embodiments, the cancer is a KEAP1-mutated cancer. In some embodiments, the cancer is lung, esophageal, kidney, head and neck, ovarian, bladder, or liver cancer. In some embodiments, the cancer is blood cancer or solid tumor. In some embodiments, the method further comprises administrating the NRF2 inhibiting agent in combination with chemotherapy or radiation, separately or together. In yet another aspect, the present disclosure provides a composition comprising a compound disclosed herein for use in vitro, in vivo, or ex vivo. Suitable compositions comprising compounds disclosed herein for use in the method of the disclosure are those described in Section I and incorporated by reference in the section in their entirety.

As described herein, NRF2 expression has been implicated in various diseases, disorders, and conditions, such as cancer. As such, modulation of NRF2 can be used for the treatment of such conditions. An NRF2 modulation agent can modulate NRF2 response or induce or inhibit NRF2 function, activity, or expression. NRF2 modulation can comprise modulating the expression, activity, or function of NRF2 on cells or modulating the quantity or number of cells that express NRF2. NRF2 modulation agents can be any composition or method that can modulate NRF2 expression, activity, or function on cells. For example, an NRF2 modulation agent can be an inhibitor or an antagonist.

One aspect of the present disclosure provides for targeting of NRF2, its co-complexed proteins, or its downstream signaling. The present disclosure provides methods of treating or preventing cancer or increasing radiosensitivity based on the discovery that an analog of pyrimethamine is 10 to 20-fold more potent in suppressing NRF2 than pyrimethamine. An NRF2 inhibiting agent can inhibit, decrease, or suppress NRF2 activity, function, signaling, downstream signaling, NRF2 protein abundance, NRF2 mRNA, or downstream targets (e.g., decreased expression of downstream NRF2 target proteins, inhibition of NRF2 protein abundance, or inhibition of downstream target gene expression).

Suppressing NRF2 activity can be useful for cancer treatment, including as agents to sensitize to traditional chemotherapy and radiotherapy.

As described herein, inhibitors of NRF2 (e.g., antibodies, fusion proteins, small molecules) can inhibit, reduce, or prevent NRF signaling, expression, activity, or function. An NRF2 inhibiting agent can be any agent that can inhibit NRF2, downregulate NRF2, or knockdown NRF2 protein or mRNA expression, activity, or function.

As another example, an NRF2 inhibiting agent can be analogs of PYR, which has been shown to be a potent and specific inhibitor of NRF2.

Inhibition of the agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

Methods and compositions as described herein can be used for the prevention, treatment, or slowing of the progression of a proliferative disease, disorder, or condition associated with NRF2, such as cancer or tumor growth. The NRF2 inhibiting agents can be used to treat cancer associated with NRF2 overexpression including as agents to sensitize to traditional chemotherapy and radiotherapy. For example, mutations in NRF2 or KEAP1 are found in many different types of cancers, but are predominately found in lung, esophageal, head and neck, ovarian, bladder, and liver cancer.

For example, cancer associated with NRF2 can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sézary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

Also provided is a process of treating, preventing, or reversing a proliferative disease, disorder, or condition associated with NRF2 in a subject in need of administration of a therapeutically effective amount of a composition of the disclosure, so as to substantially enhance radio- or chemo-sensitivity, inhibit tumor growth or cancer proliferation, slow the progress of tumor growth or cancer proliferation, or limit the development of tumor growth or cancer proliferation.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a composition of the disclosure is, for example, an amount that would cause the desired therapeutic effect in a subject while minimizing undesired side effects. In various embodiments, an effective amount of a composition of the disclosure can substantially enhance radio- or chemo-sensitivity, inhibit tumor growth or cancer proliferation, slow the progress of tumor growth or cancer proliferation, or limit the development of tumor growth or cancer proliferation.

According to the methods described herein, administration of the agent can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of compound disclosed herein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially enhance radio- or chemo-sensitivity, inhibit tumor growth or cancer proliferation, slow the progress of tumor growth or cancer proliferation, or limit the development of tumor growth or cancer proliferation.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a composition of the disclosure can occur as a single event or over a time course of treatment. For example, an NRF2 inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-I-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro¬triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, and methotrexate and pharmaceutically acceptable salts, acids or derivatives of any of the above.

Other examples of chemotherapeutic agents can include Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran (Melphalan Hydrochloride), Alkeran (Melphalan), Alimta (Pemetrexed Disodium), Aloxi (Palonosetron Hydrochloride), Ambochlorin/Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar (Irinotecan Hydrochloride), Capecitabine, CAPOX, Carac (Fluorouracil-Topical), Carboplatin, Carboplatin-Taxol, Carfilzomib, Carmubris (Carmustine), Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, Chlorambucil-prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-bevacizumab, FOLFIRI-Cetuximab, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituximab, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate).

The compounds of the disclosure can enhance radiosensitivity to radiotherapy. Radiotherapy, also called radiation therapy, can be used for the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, non-cancer cells can be capable of repairing themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of a tumor. This ensures that a higher radiation dose is given to the tumor. Healthy surrounding cells and nearby structures receive a lower dose of radiation, so the possibility of side effects is reduced.

The agents as described herein can increase the effectiveness of radiation therapy. Two types of investigational drugs are being studied for their effect on cells undergoing radiation. Radiosensitizers, as described herein, make the tumor cells more likely to be damaged, and radioprotectors protect normal tissues from the effects of radiation. Hyperthermia, the use of heat, is also being studied for its effectiveness in sensitizing tissue to radiation.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, □-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anticancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, □, and □; IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anticancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

Following contact with an effective amount of the composition of the disclosure, growth of the cancer cell is inhibited. Cell growth or proliferation can be measured in cells grown in vitro using standard cell viability or cell cytotoxicity assays (e.g., based on DNA content, cell permeability, etc.) in combination with cell counting methods (e.g., flow cytometry, optical density). Cell growth or proliferation can be measured in vivo using imaging procedures and/or molecular diagnostic indicators.

In an embodiment, contact with an effective amount of the composition selectively inhibits growth of cancer cells. As such, a composition does not appreciably kill non-cancer cells at the same concentration. Accordingly, more than 50% of non-cancer cells remain viable following contact with a composition comprising SL or a SL derivative at the same concentration. For example about 50%, about 55%, about 60%, about 65%, about 70%, about 75%, about 80%, about 85%, about 90%, about 95% or about 100% of non-cancer cells remain viable following contact with a composition comprising SL or a SL derivative at the same concentration. Or, 50%, 55%, 60%, 65%, 70%, 75%, 80%, 85%, 90%, 95% or 100% of non-cancer cells remain viable following contact with a composition at the same concentration.

Another method to measure selective inhibition of cancer cells may be via determining the LD₅₀ of the compound in the presence of cancer cells. For example, cancer cells may be cultured in the presence of a compound and the LD₅₀ may be calculated via methods standard in the art. A LD₅₀ of a compound of the disclosure may have a LD₅₀ value of about 2 μM or less. For example, a LD₅₀ of a compound of the disclosure may have a LD₅₀ value of about 2 μM or less, about 1.9 μM or less, about 1.8 μM or less, about 1.7 μM or less, about 1.6 μM or less, about 1.5 μM or less, about 1.4 μM or less, about 1.3 μM or less, about 1.2 μM or less, about 1.1 μM or less, about 1 μM or less, about 0.9 μM or less, about 0.8 μM or less, about 0.5 μM or less, about 0.4 μM or less, about 0.3 μM or less, about 0.2 μM or less, or about 0.1 μM or less. Further, a LD₅₀ of a compound of the disclosure may have a LD₅₀ value of about 0.1 μM or less. For example, a LD₅₀ of a compound of the disclosure may have a LD₅₀ value of about 0.1 μM or less, about 0.09 μM or less, about 0.08 μM or less, about 0.07 μM or less, about 0.06 μM or less, about 0.05 μM or less, about 0.04 μM or less, about 0.03 μM or less, about 0.02 μM or less, or about 0.01 μM or less.

In various embodiments, cancer cell growth may be inhibited about 0.5-fold, about 1-fold, about 2-fold, about 3-fold, about 4-fold, about 5-fold, about 8-fold, about 10-fold, or more than 10-fold relative to a reference value. In various other embodiments, cancer cell growth may be inhibited 0.5-fold, 1-fold, 2-fold, 3-fold, 4-fold, 5-fold, 8-fold, 10-fold, or more than 10-fold relative to a reference value. In other embodiments, cancer cell growth may be inhibited to such a degree that the cell undergoes cell death (via apoptosis or necrosis). Any suitable reference value known in the art may be used. For example, a suitable reference value may be cancer cell growth in a sample that has not been contacted with a composition of the disclosure. In another example, a suitable reference value may be the baseline growth rate of the cells as determined by methods known in the art. In another example, a suitable reference value may be a measurement of the number of cancer cells in a reference sample obtained from the same subject. For example, when monitoring the effectiveness of a therapy or efficacy of a composition, a reference sample may be a sample obtained from a subject before therapy or administration of a composition.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “subject” refers to a human, or to a non-human animal.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

As used herein, the terms “effective amount” or “therapeutically effective amount” of a drug used to treat a disease is an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C1-10 alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of the alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O— CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH2-cyclopropyl, —CH2-cyclobutyl, —CH2-cyclopentyl, —CH2-cyclopentadienyl, —CH2-cyclohexyl, —CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean “for example”. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Small Molecule Inhibitors of the NRF2 Transcription Factor as a Cancer Therapeutic or as a Chemo-/Radio-Sensitizing Agent

The nuclear factor E2 factor-related factor 2 (NRF2) transcription factor is aberrantly active in many human cancers. NRF2 is well-established to drive chemo- and radiation resistance in human cancer. Beyond cancer, NRF2 activation has been reported to govern additional human diseases. The present example provides Pyrimethamine and analogs thereof function to decrease NRF2 mRNA levels, decrease NRF2 protein levels, and NRF2 activity (e.g., reduced downstream NRF2 target gene expression) through inhibition of dihydrofolate reductase (DHFR).

### Results

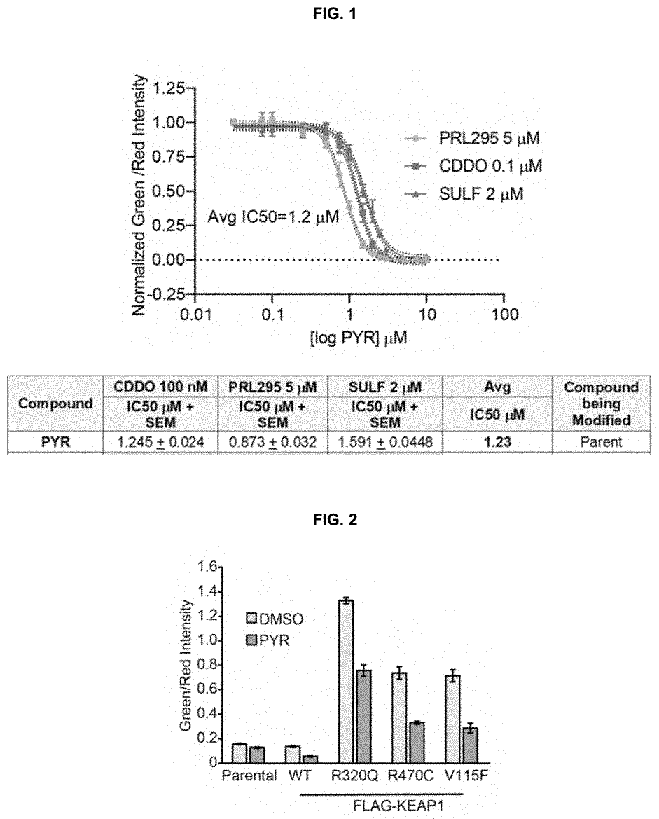

PYR decreases NRF2 mRNA and protein resulting in lower pathway activity and proliferation: Pyrimethamine (PYR) was discovered as a NRF2 inhibitor from a screen of the Prestwick compound library. Using an NRF2 activity-dependent lung cancer reporter cell line, H1299-NQO1-YFP cells, the IC₅₀ was defined of PYR against three NRF2/KEAP1 pathway activators (CDDO-ME (0.1 mM final), SULF (2 μM final), or PRL-295 (5 μM)). On average, PYR suppresses these agonists at an IC₅₀ of 1.2 μM (Table 1, FIG. 1). FIG. 1 shows the IC₅₀ curve calculated at one time point for PYR against the different NRF2 activators. To demonstrate PYR negatively regulates NRF2 activity without the use of small molecule activators, the same cell reporter line was used and mutant KEAP1 was overexpressed. Indeed, PYR repressed mutant KEAP1 activation of the pathway as shown in FIG. 2.

TABLE 1
Analog IC50 results in H1299-NQO1-YFP cells vs activators.

| | | | | | Compound |
|---|---|---|---|---|---|
| | CDDO 0.1 μM | PRL295 5 μM | SULF 2 μM | Avg | being |
| Compound | IC₅₀ μM ± SEM | IC₅₀ AM ± SEM | IC₅₀ μM ± SEM | IC₅₀ μM | Modified |
| PYR | 1.245 ± 0.024 | 0.873 ± 0.032 | 1.591 ± 0.0448 | 1.23 | Parent |
| WCDD101 | | | | >20 | PYR |
| WCDD103 | 16.86 ± 2.563 | 9.99 ± 0.0737 | 12.46 ± 0.798 | 13.10 | PYR |
| WCDD104 | 0.103 ± 0.001 | 0.061 ± 0.003 | 0.129 ± 0.003 | 0.098 | PYR |
| WCDD105 | 5.115 ± 0.074 | 3.743 ± 0.4988 | 3.279 ± 0.006 | 4.04 | PYR |
| WCDD106 | | | | >20 | PYR |
| WCDD107 | | | | >20 | PYR |
| WCDD108 | 16.85 ± 0.208 | 15.21 ± 1.295 | 12.56 ± 0.1033 | 14.87 | PYR |
| WCDD110 | | | | >20 | PYR |
| WCDD111 | 1.105 ± 0.016 | 1.511 ± 0.0329 | 0.910 ± 0.010 | 1.175 | PYR |
| WCDD112 | 19.84 ± 0.338 | 10.83 ± 0.620 | 10.92 ± 0.0655 | 13.86 | PYR |
| WCDD113 | | | | >20 | PYR |
| WCDD114 | 0.134 ± 0.014 | 0.079 ± 0.002 | 0.111 ± 0.009 | 0.108 | WCDD104 |
| WCDD115 | 0.052 ± 0.0001 | 0.060 ± 0.001 | 0.057 ± 0.003 | 0.057 | WCDD104 |
| WCDD118 | | | | >5 | WCDD104 |
| WCDD119 | 0.614 ± 0.021 | 0.552 ± 0.003 | 0.557 ± 0.03 | 0.57 | WCDD104 |
| WCDD120 | | | | >10 | WCDD104 |
| WCDD121 | | | | >5 | WCDD104 |
| WCDD122 | | | | >5 | WCDD104 |
| WCDD123 | | | | >20 | WCDD104 |
| WCDD125 | | | | >20 | WCDD104 |
| WCDD126 | 0.461 ± 0.017 | 0.683 ± 0.002 | 0.397 ± 0.011 | 0.514 | WCDD104 |
| WCDD127 | 0.469 ± 0.002 | 0.444 ± 0.002 | 0.454 ± 0.006 | 0.456 | WCDD104 |
| WCDD128 | 0.244 ± 0.006 | 0.334 ± 0.004 | 0.163 ± 0.002 | 0.248 | WCDD104 |
| WCDD133 | | | | >20 | WCDD104 |
| WCDD133a | | | | >20 | WCDD104 |
| WCDD134 | 1.84 ± 0.196 | 1.84 ± 0.196 | 1.88 ± 0.024 | 1.43 | WCDD104 |
| WCDD139 | 0.206 ± 0.0014 | 0.106 ± 0.003 | 0.097 ± 0.003 | 0.14 | WCDD115 |
| WCDD135 | | | | >20 | WCDD115 |
| WCDD136 | | 0.153 ± 0.002 | | 0.153 | WCDD115 |
| WCDD137 | | 4.84 ± 0.284 | | 4.84 | WCDD115 |

| | | |
|---|---|---|
| WCDD138 | Synthesized, not tested | WCDD115 |

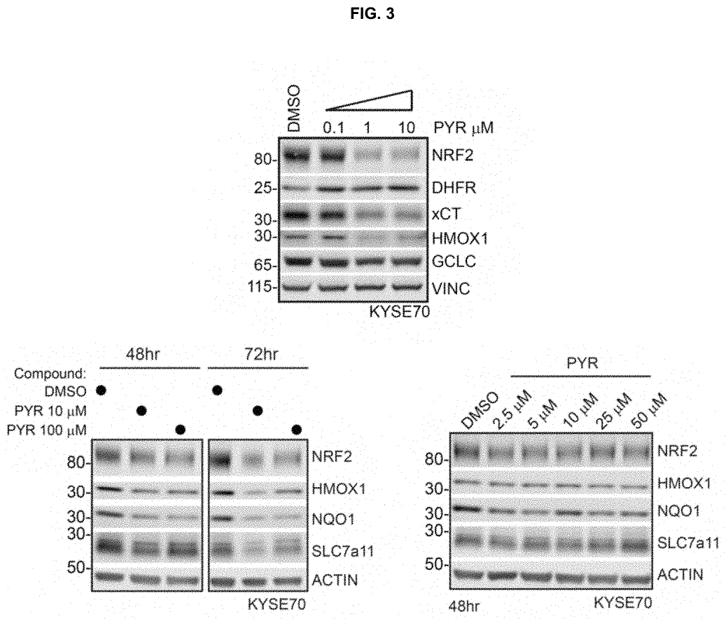

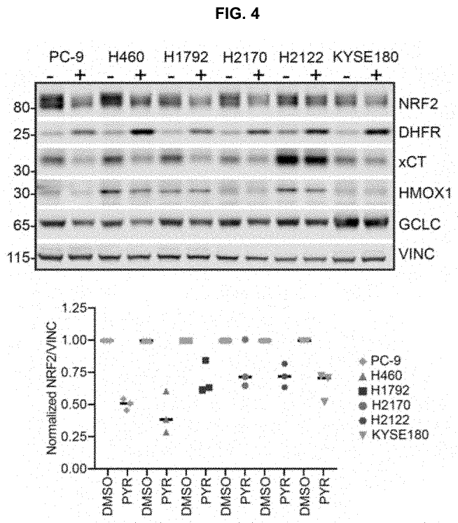

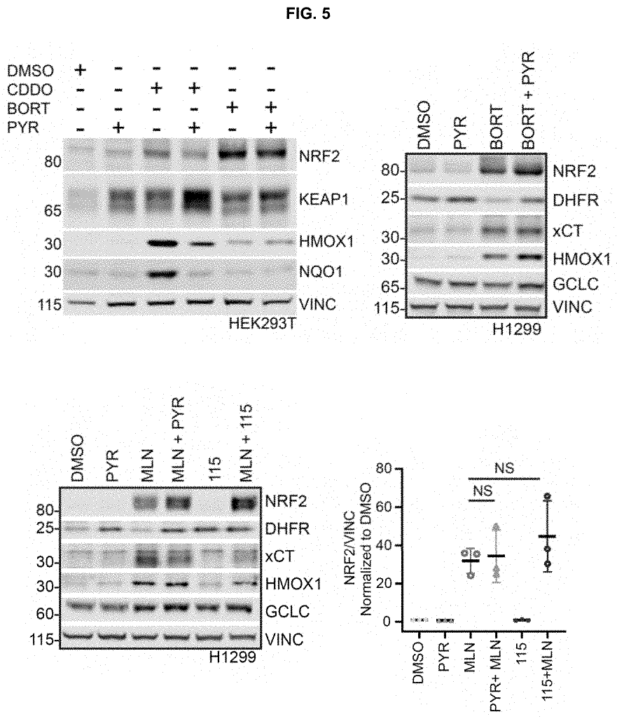

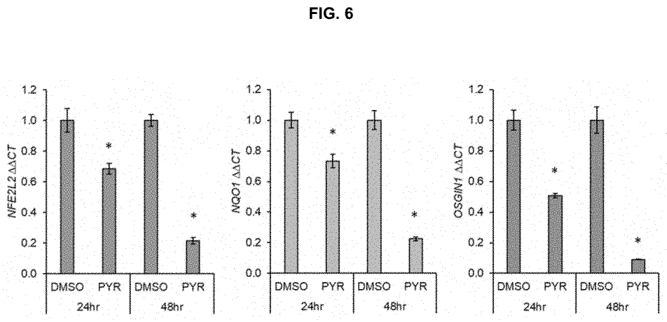

To better understand how PYR was suppressing NRF2 activity, the consequence of PYR administration on NRF2 protein abundance was investigated. It was found that PYR decreases NRF2 protein abundance in both mutant/active NRF2 cells (FIG. 3 and FIG. 4) and in wild type NRF2/KEAP1 cells (HEK293T) treated with pathway activators (FIG. 5). As expected, PYR also resulted in decreased expression of downstream NRF2 target proteins in all cases (FIG. 3-FIG. 5). Importantly, HEK293T cells treated with Bortezomib, a proteasome inhibitor, prevented PYR from decreasing NRF2, suggesting that PYR relies on proteasomal activity to inhibit NRF2 (FIG. 5, in both HEK293T and H1299 cells). Since NRF2 protein levels were decreased with PYR treatment, the effect on NRF2 mRNA levels was investigated, and indeed PYR treatment resulted in abrogated NRF2 mRNA and NRF2 target gene mRNA decrease starting at 24 hours becoming much more pronounced at 48 hours (FIG. 6).

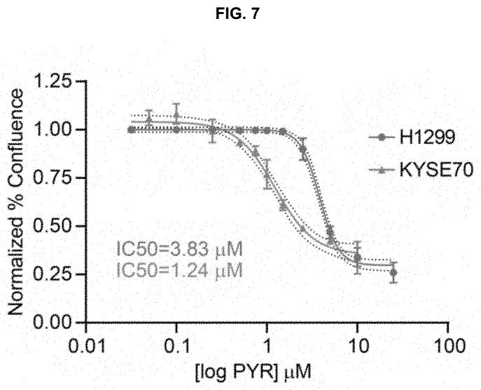

To see if the decrease in NRF2 mRNA, protein, and activity negatively impacted cell proliferation, two cell lines were used and treated with different doses of PYR for several days. It was found that the NRF2 addicted cell KYSE70 was impacted more by PYR on its growth than H1299 a cell line with an intact NRF2 pathway (FIG. 7). KYSE70 cells had an IC₅₀ of 1.24 uM compared to H1299 which had an IC₅₀ of 3.83 uM when comparing percent confluence at 96 hours post treatment.

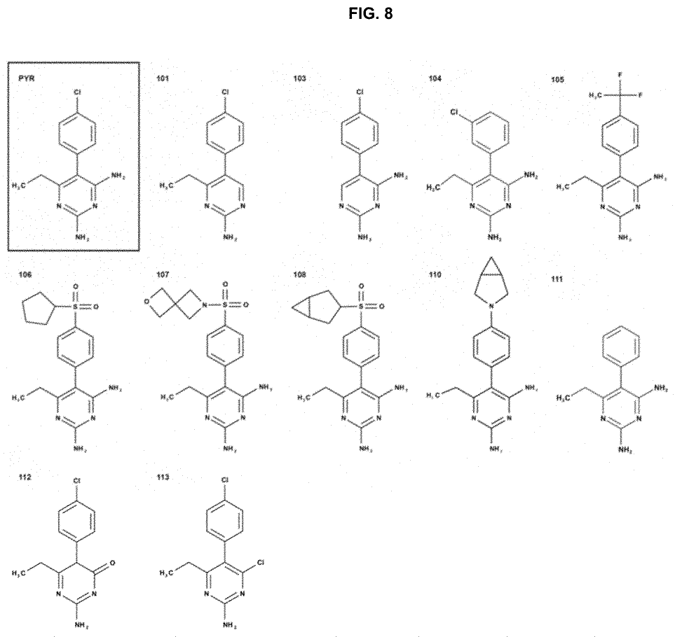

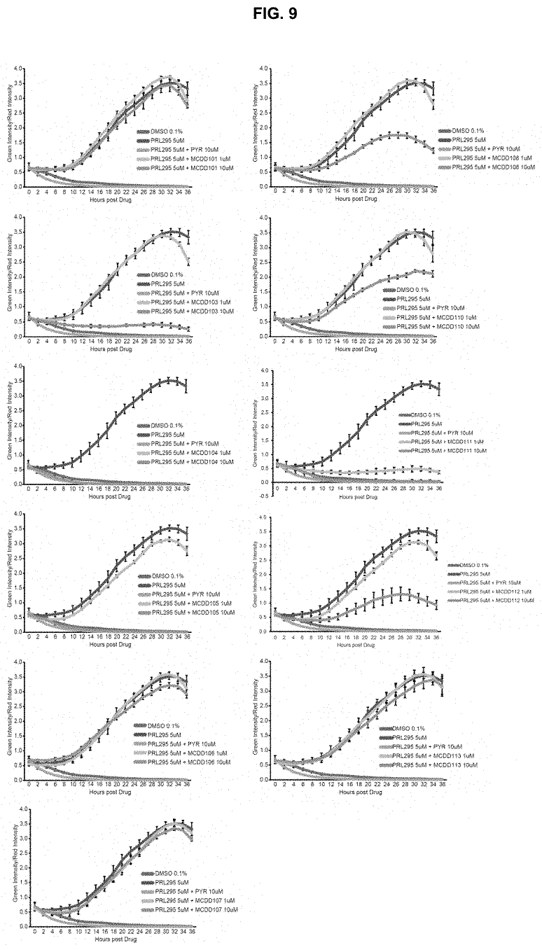

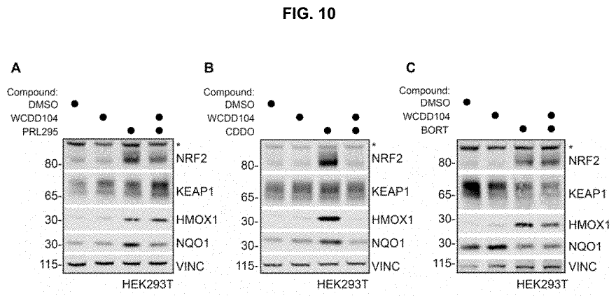

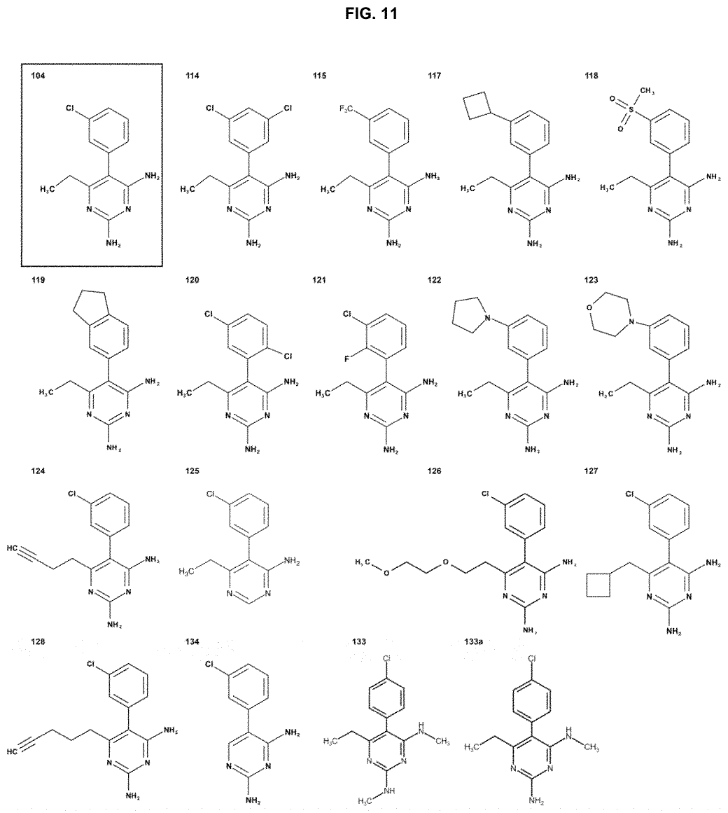

PYR analogs: Next, more potent NRF2 inhibitors based on the PYR parent compound were developed and tested. In a first round of structure activity relationship, several analogues were developed (FIG. 8). These were then tested in the same H1299-NQO1-YFP system against the NRF2 activating drug PRL295 using two doses (FIG. 9). Based on these results only analogs that demonstrated some decrease in the reporter with the 10 μM dose went on for further testing. The resulting experiments revealed that only analogs 111 and 104 had comparable or lower IC₅₀s to the parental PYR compound, respectively (Table 1). Testing analog 104 in HEK293T against the different pathway activators also confirmed inhibition of NRF2 protein abundance and downstream target gene expression (FIG. 10A-10B). Co-treatment of analog 104 with Bortezomib also demonstrated a proteasomal requirement for activity (FIG. 10C).

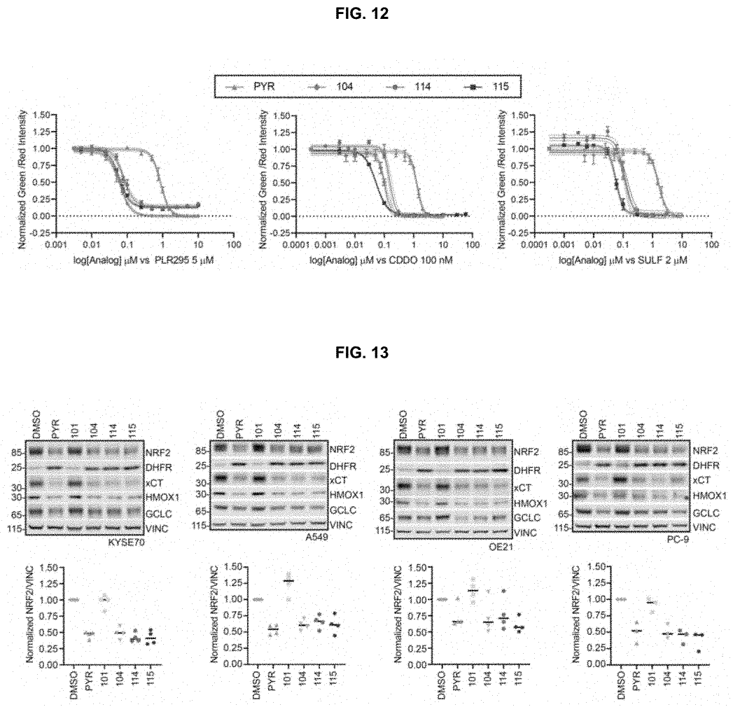

In a second round of SAR, analog 104 was further modified (FIG. 11). Some of these changes were to the chloride group again, testing more additions or bulky groups to this ring or adding bulk/chains to the original ethyl group. Excitingly, this round of changes revealed several promising compounds. First, adding a 5 membered ring to where the chloride group was, made analog 119 comparable to PYR, (IC₅₀ 0.5 μM vs 1.2 μM, respectively Table 1). Second adding a second chloride group on the other side of the ring did not negatively affect the activity as this analog was comparable to 104, 114 (IC₅₀ 0.1 μM vs 0.1 μM, respectively (FIG. 12, Table 1). Third, changing the chloride group to a completely fluorinated carbon made analog 115 a little bit better than 104 (IC₅₀ 0.05 μM vs 0.1 μM, respectively) (FIG. 12, Table 1). Interestingly there were several analogs with bulky or long chains, which had IC₅₀s around 0.5 μM (Analogs 126-128) suggesting larger groups at that site would be tolerated such as biotin (FIG. 11, Table 1). Additional methyl chains off of amine groups were not tolerated at all (analogs 133-133a, FIG. 11, Table 1).

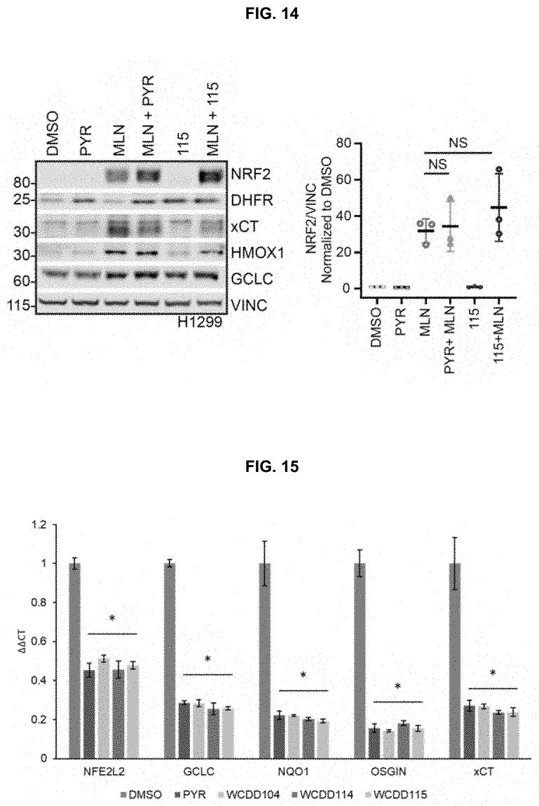

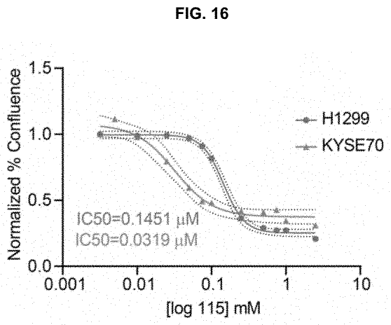

The best analogs to PYR, 104, 114, and 115 were then tested in NRF2 mutated/upregulated cell lines cells for 48 hours and found indeed these analogs decrease NRF2 protein abundance and as well as downstream targets at a dose of 1 μM vs 10 μM used for PYR. Also of note, analog 101 was used as a negative control as 101 did not have any effect on NRF2 activity in the IC₅₀ experiments at the doses used (FIG. 13). Similarly, to BORT co-treatment inhibiting neddylation of CUL3, part of the E3 complex responsible for ubiquitinating NRF2, was required for both PYR and 115 to inhibit NRF2 protein levels (FIG. 14). As with PYR, the best analogs decreased both NRF2 mRNA and downstream target genes (FIG. 15). Lastly in an initial experiment, analog 115 demonstrated to be more potent in suppressing the proliferation of the NRF2 addicted KYSE70 cell line compared to the wild type H1299 cells (IC₅₀ 0.0319 μM vs 0.1451 μM respectively) (FIG. 16).

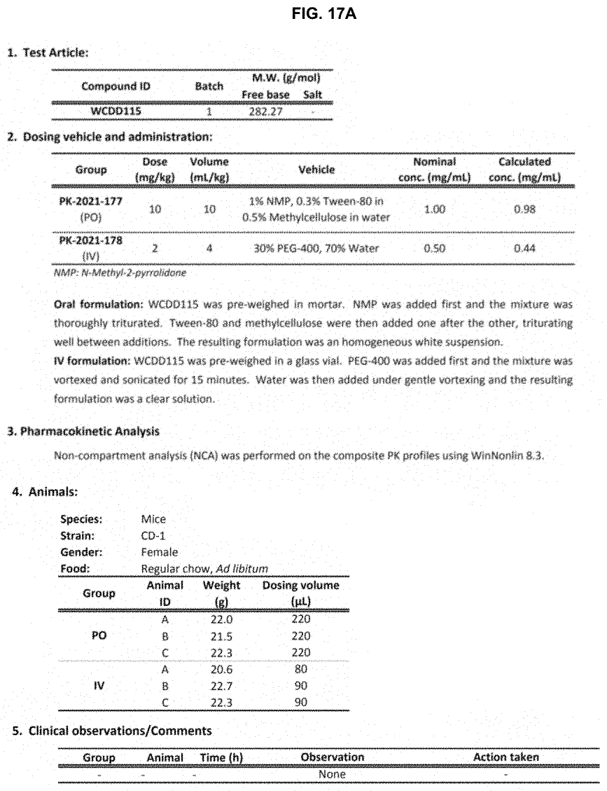

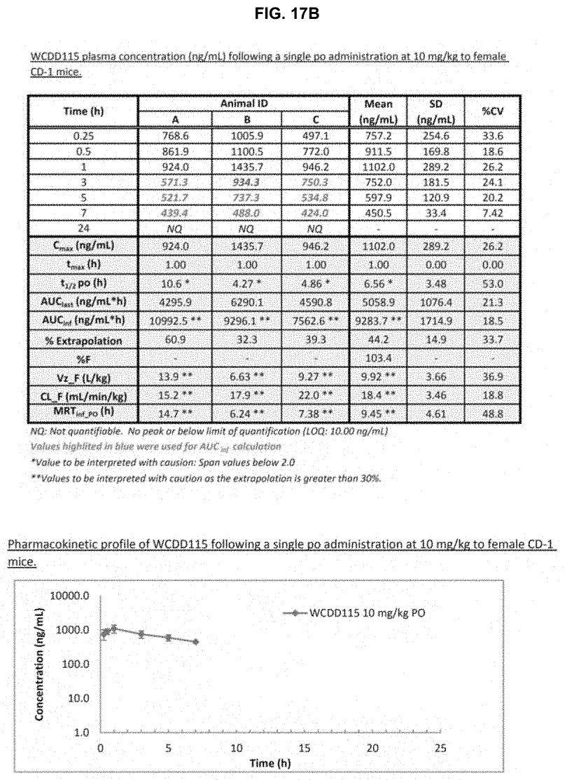

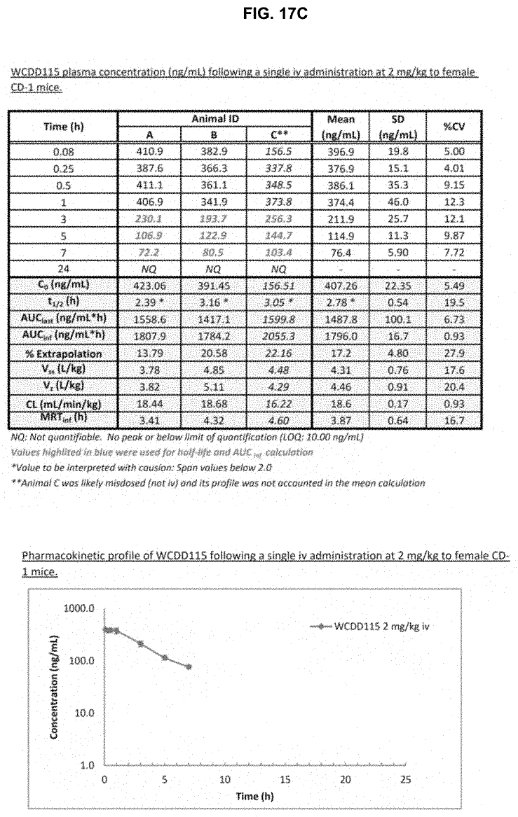

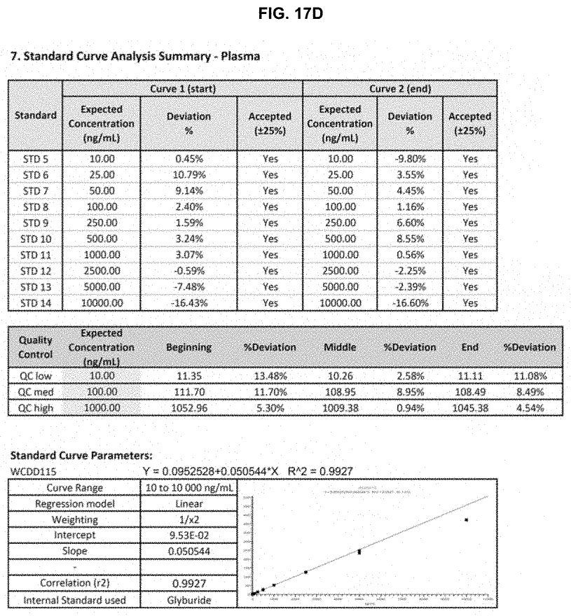

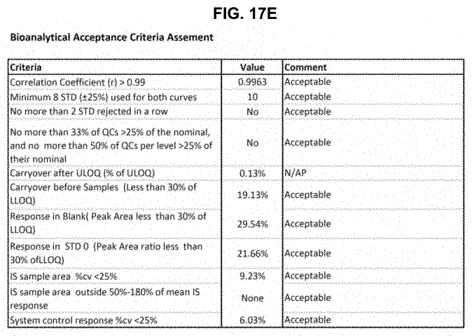

Since analog 115 has a 10-fold improvement over PYR, the pharmacokinetic profile of this compound was tested in mice (FIG. 17). Two tests were conducted, on for a single oral dose of 10 mg/kg and for an intravenous 2 mg/kg dose in female CD-1 mice (n=3 for each group). In both administration methods, no clinical observations were made suggesting 115 was well tolerated. Oral dose of analog 115 had higher plasma levels as compared to the IV administration, with about 450+/−33.4 ng/ml compared to 76.4+/−5.90 ng/ml at hour 7. Analog 115 was unquantifiable at 24 hours post dosing in either scenario (FIG. 17). Overall, 115 was well tolerated and had decent plasma retention time.

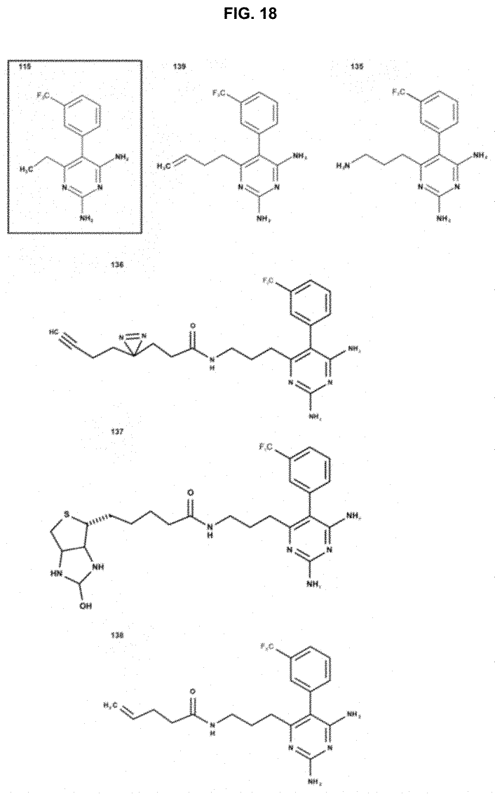

Lastly, analogs to 115 which have good IC₅₀s and a tag for mechanistic downstream experiments were generated (FIG. 18). In one example, an intermediate of 139, has been tested for generating these compounds. Analog 139, with the longer chain in replace of the original ethyl group still had a very good average IC₅₀ of 0.14 μM (Table 1). Analog 136 with a photo-linkable dizarine group replacing the original ethyl group also had a good IC₅₀ of 0.153 μM, meaning future cross-linking experiments can be performed with this analog (Table 1).

DHFR Activity: Since PYR was originally found to be a DHFR inhibitor of plasmodia, but the literature was unclear regarding its activity against human DHFR, western blots were probed for DHFR protein as it is known that when it is inhibited it has a feedback mechanism to stabilize its own mRNA and protein. Indeed, it was found at the doses that were used, DHFR protein was stabilized (FIGS. 3-5,13, 14). Interestingly in FIG. 13 where analog 101 was used as a negative control due to its lack of effect on NRF2 activity, it was observed that DHFR was not stabilized, suggesting that DHFR was not inhibited. Thus, it was hypothesized that inhibition of DHFR activity was necessary for PYR and analogs to suppress NRF2 signaling.

Using an in vitro enzymatic assay, it was found that PYR does inhibit human DHFR (hDHFR) with an IC₅₀ of 4.49 μM whereas 115 inhibits at 0.114 μM (Table 2). MTX was used as a positive control and inhibited within kit range of 0.00597 μM. Analog 115 was shown to inhibit hDHFR several fold better than PYR suggesting that perhaps 115 was better than PYR due to its ability to better target hDHFR

TABLE 2
Human DHFR enzymatic assay IC₅₀ results

| | hDHFR3E-3 Units |
|---|---|
| Compound | IC₅₀ μM + SEM |
| MTX | 0.00597 ± 0.000673 |
| PYR | 4.49 ± 0.635 |
| 115 | 0.144 ± 0.0224 |

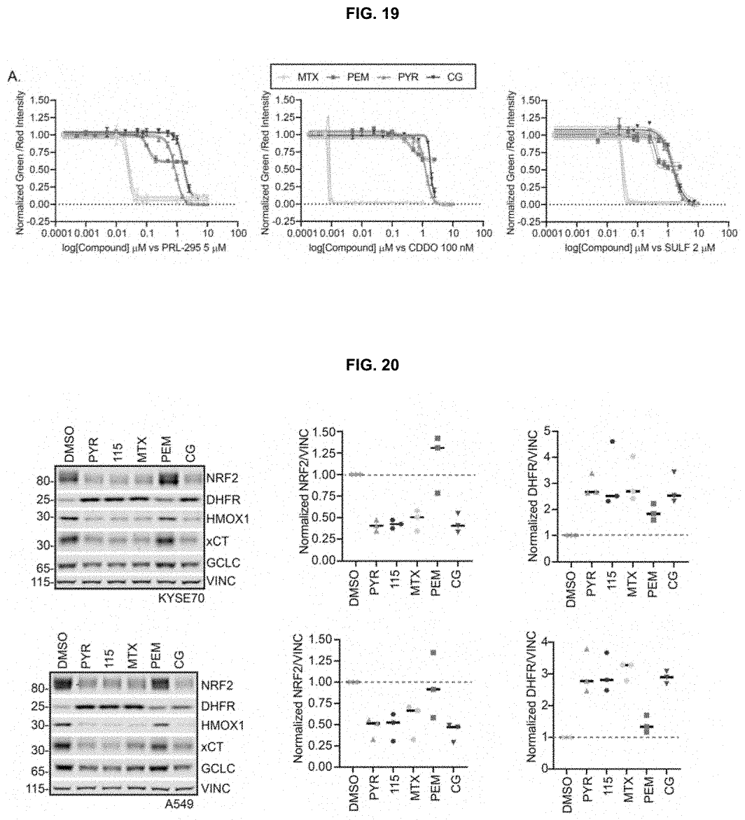

To see if other DHFR inhibitors can affect NRF2 signaling, IC₅₀ analysis was performed in H1299-NQO1-YFP cells as before with MTX, PEM, and CG. MTX is a well characterized hDHFR inhibitor, PEM can inhibit hDHFR but is a better TS inhibitor, and finally CG is the active form of which is a similar plasmodia DHFR inhibitor to PYR. It was found that all inhibitors did suppress NRF2 activity to different degrees (FIG. 19, Table 3). CG had an IC₅₀ comparable to PYR (1.695 μM vs 1.23 μM respectively). MTX had the most robust suppression with an IC₅₀ of 0.0202 μM. Interestingly, PEM had only partial suppression of NRF2 activity at the doses tested.

TABLE 3
DHFR IC₅₀ results in H1299-NQO1-YFP cells vs activators.

| Comp- | CDDO 100 nM | PRL295 5 μM | SULF 2 μM | Avg |
|---|---|---|---|---|
| ound | IC₅₀ μM ± SEM | IC₅₀ μM ± SEM | IC₅₀ μM ± SEM | IC₅₀ μM |
| PYR | 1.245 ± 0.024 | 0.873 ± 0.032 | 1.591 ± 0.0448 | 1.23 |
| MTX | 0.0008 ± 3.4E-05 | 0.0240 ± 0.0024 | 0.0358 ± 0.0031 | 0.0202 |
| PEM | 0.334 ± 0.03 | 0.1121 ± 0.0068 | 0.382 ± 0.0374 | 0.2760 |
| CG | 1.82 ± 0.085 | 1.57 ± 0.008 | 1.503 ± 0.0466 | 1.695 |

Numbers italicized indicate incomplete inhibition

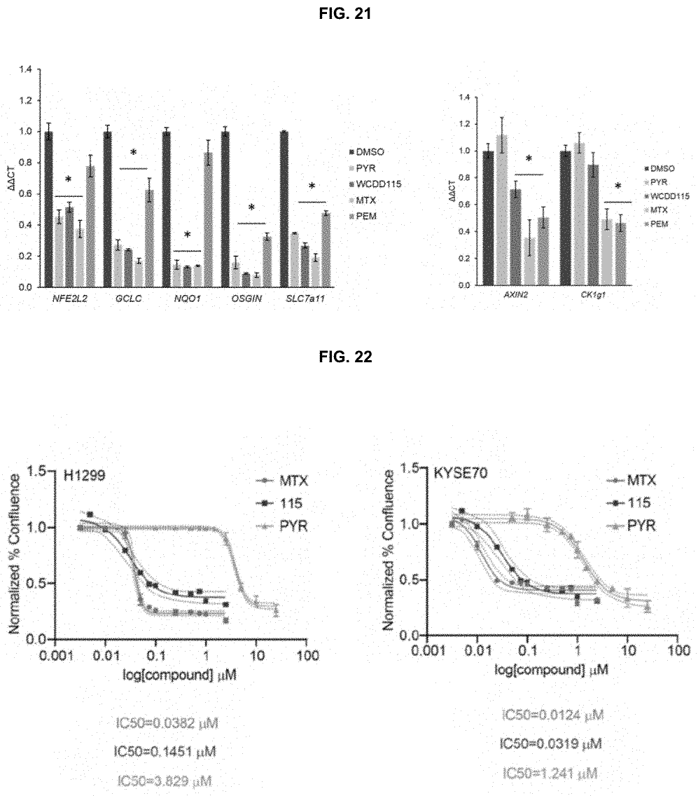

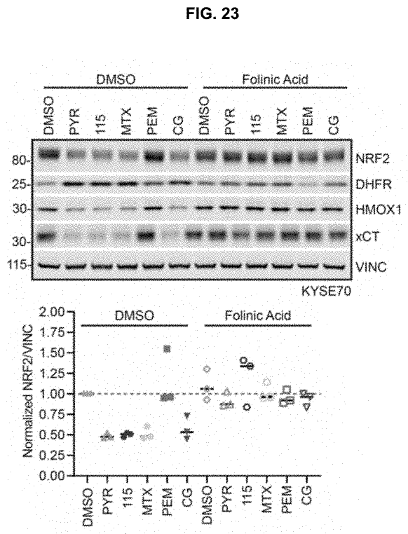

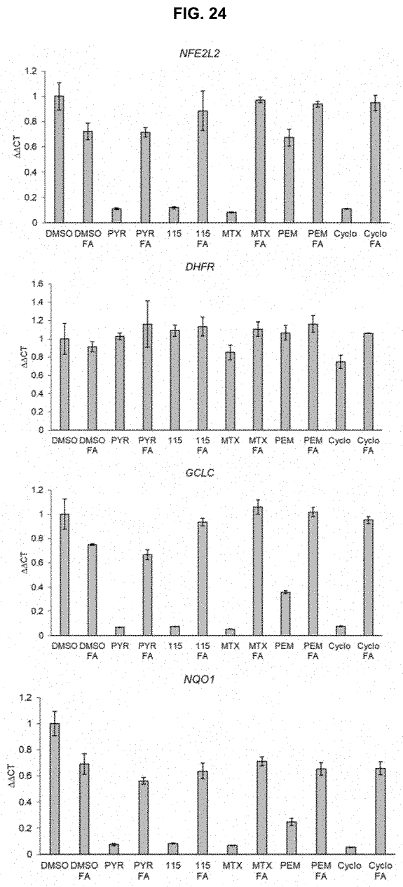

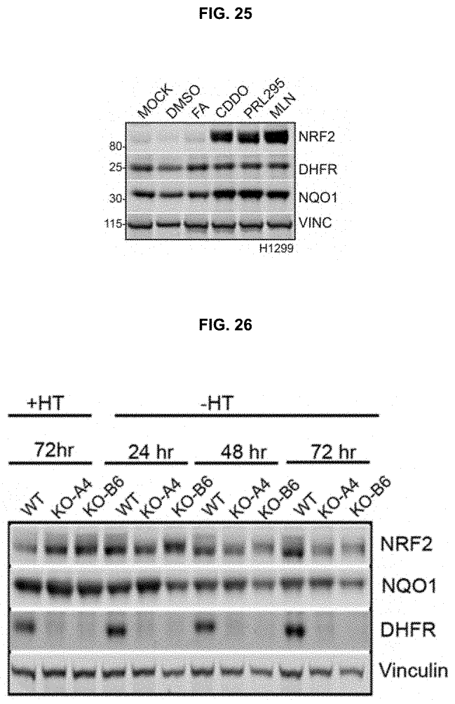

Next, whether these DHFR inhibitors also decreased NRF2 protein and mRNA was investigated and indeed it was found that MTX and CG decreased NRF2 protein and mRNA, but PEM did not (FIG. 20 and FIG. 21). Both MTX and PEM decreased AXIN2 and CK1γ1 mRNA which are not target genes of NRF2 suggesting that these inhibitors may have off target or nonspecific activity compared to PYR and 115, although 115 does have some minimal effect on AXIN2 (FIG. 21). Since MTX does decrease NRF2 protein and mRNA, a belt with possible off target affects, we next wanted to test MTX's ability to suppress proliferation in a NRF2 wild type cell and a NRF2 addicted cell line as in FIGS. 7 and 16. Interestingly, even though MTX has a much more robust IC₅₀ for our reporter assay (Table 3), we found that it was comparable to analog 115 in suppressing cell proliferation (IC₅₀ 0.0124 μM and IC₅₀ 0.0319 μM respectively in KYSE70 cells) (FIG. 22). Since DHFR is the rate limiting step to the folate pathway, rescue of DHFR inhibition was tested by co-treatment with a downstream metabolite mimic Folinic Acid (FA). Both NRF2 protein and mRNA were rescued with co-treatment in KYSE70 cells after 48-hour treatment (FIG. 23 and FIG. 24). As a control, FA alone was also tested in H1299 cells and no stabilization of NRF2 (FIG. 25) was observed, ruling out FA acting as a NRF2 stabilizer.

To determine if loss of DHFR protein was necessary for the suppression of NRF2 two CRISPRi DHFR KYSE70 cell lines were generated. These cells require treatment with HT (hypoxanthine and thymidine) to continue to grow otherwise the cells die. When HT is removed, NRF2 protein levels go down in the CRISPRi DHFR KO cells suggesting that PYR and analogs are indeed working through DHFR inhibition (FIG. 26).

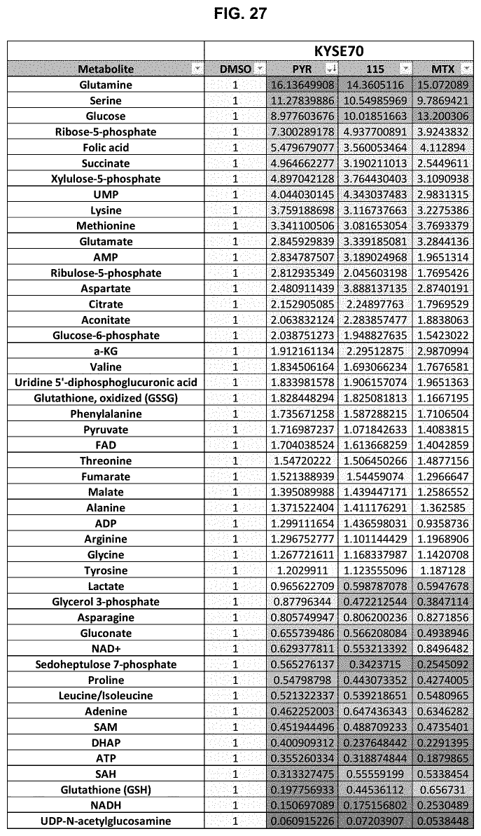

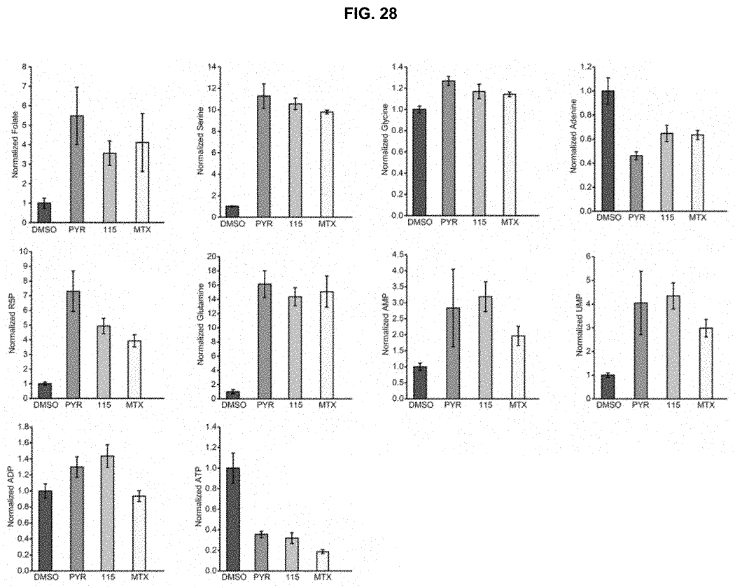

To better understand the effects of inhibition with PYR, MTX and 115 metabolomics analysis was performed on KYSE70 cells treated for 48 hours (heatmap summary FIG. 27). Folate levels were found to increase >4 fold in all treated samples as compared to DMSO (FIG. 28). Similarly, metabolites in the folate pathway were also increased, like Serine and Glycine that is normally made from Serine in this pathway is mostly unchanged. Adenine the precursor to AMP is decreased suggesting decreased purine synthesis. Similarly, R5P and Glutamine are elevated, both of which can feed into generating nucleotides indicating a halt in this production. AMP, UMP and to a lesser extent ADP are all up whereas ATP is very low in treated cells (FIG. 28).

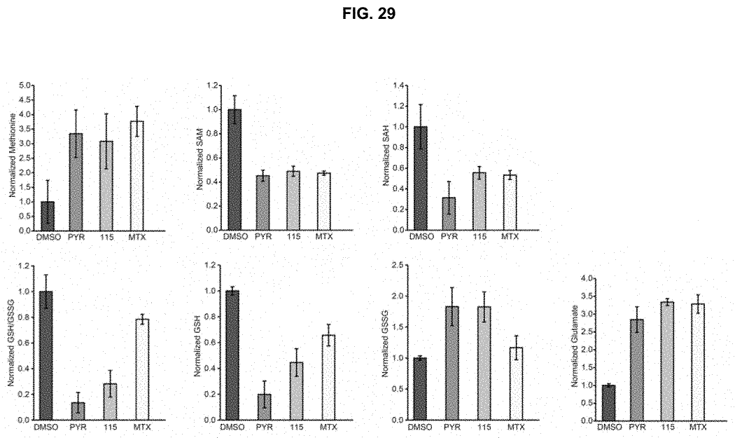

One carbon metabolism was also stalled as seen by high levels of Methionine, but low levels of SAM and SAH (FIG. 28). And lastly, redox metabolites were investigated and found that reduction potential in the form of glutathione ratios was decreased in PYR and 115 treated cells, but to a lesser extent with MTX again suggesting that MTX has other effects in cells that are NRF2 independent (FIG. 29).

Taken together, PYR and analogs through inhibition of DHFR decrease NRF2 mRNA and protein abundance resulting in decreased NRF2 activity (activity reporter data, metabolomics) and cell proliferation. Other DHFR inhibitors also can affect NRF2 signaling but may have potential off target affects.

### Methods

Tissue culture: H1299-NQO1-YFP, H1299, KYSE70, A549, H460, PC-9, H1792, H2170, H2122, KYSE180, OE21 cells were cultured in RPMI supplemented with 10% FBS and grown at 37° C. with 5% CO₂. HEK293T cells were cultured in DMEM supplemented with 10% PBS and grown in the same incubator conditions as described above.

H1299-NQO1-YFP cells were transfected with WT, R320Q, R470C, or V115F FLAG-KEAP1 constructs using Lipofectamine 2000. The next day cells were treated with either DMSO or 10 μM PYR and imaged for another 24 hours ever 2 hours in an IncuCyte S3. Calculations are detailed below. This was performed in biological triplicate and error bars are SD.

IncuCyte and IC₅₀ calculations: In a 96 well plate, 3,000 H1299-NQO1-YFP cells were plated, and the following day treated with one of three activators, CDDO-ME (0.1 μM final), SULF (2 μM final), or PRL-295 (5 μM). Co-treating with one of these activators a dose curve of PYR, PYR analogs, or other DHFR inhibitors was performed. Readings were taken every 2 hours for at least 24 hours. For proliferation assay, H1299 or KYSE70 cells were similarly plated (1,500 cells and 6,000 cells respectively) and treated the following day with PYR. Readings were also taken every 2 hours for 96 hours in and IncuCyte S3.

YFP total intensity (correlating to NRF2 activity) was normalized to NLS-mCherry (cell number) total intensity to give a ratio (YFP/mCherry). Ratios at one time point at least 24 hours post compounds were added were used. These numbers were then plotted in Graphpad Prism to calculate IC₅₀s. Three biological replicates were performed for each PYR analog that met an estimated IC₅₀ of less than 5 μM from a smaller initial dose curve experiment performed similarly as described. From three biological replicates, percent confluence was used for proliferation assays to calculate IC₅₀s, calculations performed using Graphpad Prism.

Western Blots: KYSE70, HEK293T cells were plated and 24 hours later treated with compounds (see figure legends for details). Either at 24 or 48 hours later, cells were scrapped in RIPA buffer (0.2% deoxycholate, 0.1% SDS, 1% NP40, 150 mM NaCl, 50 mM Tris pH 7.4, 2 mM EDTA) supplemented with protease and phosphatase inhibitors, cleared at 14000×g for 15 minutes at 4° C., and supernatants were quantified by BCA assay (Pierce, 23225). Protein samples (20 μg) were prepared with 1×LDS buffer with 100 mM DTT, ran on NuPAGE 4-12% Bis-tris gels, and transferred onto a 0.45 μM nitrocellulose membrane (Thermo, 88018). All blots were blocked in 5% milk in TBST for 30 minutes. After incubation of the primary antibody overnight at 4° C. and washed three times with 1×TBST, blots were incubated with LI-COR secondary antibodies for 1-2 hours, washed in 1×TBST thrice, and then imaged via a LI-COR Odyssey instrument (Li-COR, Lincoln, Nebr.). Antibodies are listed in Table 4.

TABLE 4
Antibodies used

| Antibody | Company | Cat # | Dilution | Primary |
|---|---|---|---|---|
| NRF2 | Abeam | Ab135570 | 1 to 1000 | Primary |
| DHFR | Cell Signaling | 45710 | 1 to 1000 | Primary |
| xCT/SLC7a11 | Cell Signaling | 12691 | 1 to 1000 | Primary |
| HMOX1 | Abeam | Ab13248 | 1 to 1000 | Primary |
| GCLC | Abeam | Ab190685 | 1 to 1000 | Primary |
| NQ01 | Novus | NB2000-209 | 1 to 1000 | Primary |
| VINCULIN | Santa Cruz | Sc-25336 | 1 to 3000 | Primary |
| Anti-Rabbit | LI-COR | 92632213 | 1 to 10000 | Secondary |
| 800 | | | | |
| Anti-Mouse | LI-COR | 92668072 | 1 to 10000 | Secondary |
| 700 | | | | |

qRT-PCR: Total RNA was extracted using PureLink RNA kit following the manufacturers protocol. Primers for qPCR were designed using the National Center for Biotechnology Information's (NCBI) Primer-BLAST platform. For primer sequences, see Table 2. RNA was quantified using a Nanodrop One (Thermo, Waltham, Mass.), and the reverse transcription reaction was performed using 2 μg of RNA with the iSCRIPT Clear kit with DNAse (Biorad, 170-8891). For the qRT-PCR, PowerUP SYBR Green (Thermo, A25741) was used, and data were analyzed on an AB QuantStudio 6 Flex Real Time PCR machine (Applied Biosystems, Foster City, Calif.). ΔCT values were normalized to housekeeping gene RPL13a and then normalized to DMSO for final ΔΔCT value. Primers are listed in Table 5.

TABLE 5
qPCR primers used.
qPCR Primers

| | Product | | |
|---|---|---|---|
| Gene | Size | Direction | Sequence 5′ → 3′ |
| RPL13 | 157 | FWD | (SEQ ID NO: 1) |
| | | | CATAGGAAGCTGGGAGCAAG |
| | | REV | (SEQ ID NO: 2) |
| | | | GCCCTCCAATCAGTCTTCTG |
| NFE2L2 | 106 | FWD | (SEQ ID NO: 3) |
| | | | AGTGGATCTGCCAACTACTC |
| | | REV | (SEQ ID NO: 4) |
| | | | CATCTACAAACGGGAATGTCTG |
| GCLC | 96 | FWD | (SEQ ID NO: 5) |
| | | | GAGGTCAAACCCAACCCAGT |
| | | REV | (SEQ ID NO: 6) |
| | | | TGTTAAGGTACTGAAGCGAGGG |
| SLC7a11 | 165 | FWD | {SEQ ID NO: 7) |
| | | | TGTGTGGGGTCCTGTCACTA |
| | | REV | (SEQ ID NO: 8) |
| | | | CAGTAGCTGCAGGGCGTATT |
| NQO1 | 117 | FWD | (SEQ ID NO: 9) |
| | | | TGCTGCAGCGGCTTTGAAGAAG |
| | | REV | (SEQ ID NO: 10) |
| | | | GCAGGGTCCTTCAGTTTACCTGTG |
| OSGIN1 | 156 | FWD | (SEQ ID NO: 11) |
| | | | GAGCCTGGCACTCCATCGAA |
| | | REV | (SEQ ID NO: 12) |
| | | | CCCTGTAGTAGTGGGCGATG |
| CSNK1g1 | 115 | FWD | (SEQ ID NO: 13) |
| | | | CCTCATTTGCGCCTTGCAG |
| | | REV | (SEQ ID NO: 14) |
| | | | CTCCGGGAGATGAAAAACCA |
| DHFR | 149 | FWD | (SEQ ID NO: 15) |
| | | | AGAATGACCACAACCTCTTCAGT |
| | | REV | (SEQ ID NO: 16) |
| | | | CCTTGTGGAGGTTCCTTGAG |

Mouse study with Analog 115: All mouse work was performed by Paraza Pharma, Inc. All pertinent information and methods are listed in FIG. 17.

Metabolomics Analysis: KYSE70 cells (500,000 cells) were plated in 6 cm dishes and the next day the cells were treated with either DMSO, 10 μM PYR, or 0.1 μM MTX. After 48 hours, 1 ml of media was aliquoted into a clean Eppendorf and kept on ice, and the rest of the media was removed from these dishes. The cells were then washed in water twice and then scrapped in ice cold methanol.

TABLE 6
NRF2 inhibitors.

| | | | | Avg |
|---|---|---|---|---|
| | | Derivative | | IC50 |
| Chemical Formula | WU Name | of: | IUPAC Name | μM |
| 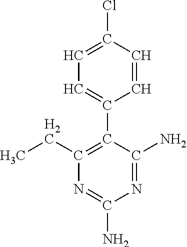 [SMILES: CCc1nc(N)nc(N)c1-c1ccc(Cl)cc1] | PYR | Parent | 5-(4-chlorophenyl)-6- ethylpyrimidine-2,4-diamine | 1.23 |
| 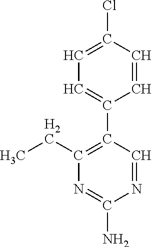 [SMILES: CCc1nc(N)ncc1-c1ccc(Cl)cc1] | WCDD101 | PYR | 5-(4-chlorophenyl)-4- ethylpyrimidin-2-amine | >20 |
| 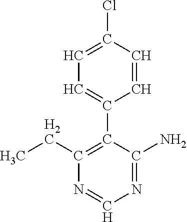 [SMILES: CCc1ncnc(N)c1-c1ccc(Cl)cc1] | WCDD102 | PYR | 5-(4-chlorophenyl)-6- ethylpyrimidin-4-amine | |
| 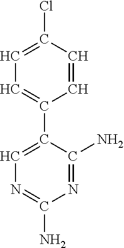 [SMILES: Nc1ncc(-c2ccc(Cl)cc2)c(N)n1] | WCDD103 | PYR | 5-(4-chlorophenyl)pyrimidine-2,4- diamine | 13.1 |
| 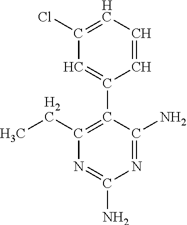 [SMILES: CCc1nc(N)nc(N)c1-c1cccc(Cl)c1] | WCDD104 | PYR | 5-(3-chlorophenyl)-6- ethylpyrimidine-2,4-diamine | 0.098 |
| 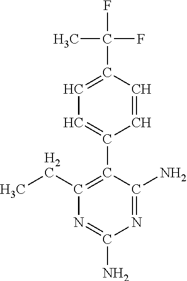 [SMILES: CCc1nc(N)nc(N)c1-c1ccc(C(C)(F)F)cc1] | WCDD105 | PYR | 5-(4-(1,1-difluoroethyl)phenyl)-6- ethylpyrimidine-2,4-diamine | 4.04 |
| 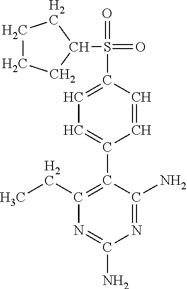 [SMILES: CCc1nc(N)nc(N)c1-c1ccc(S(=O)(=O)C2CCCC2)cc1] | WCDD106 | PYR | 5-(4-(cyclopentylsulfonyl)phenyl)- 6-ethylpyrimidine-2,4-diamine | >20 |
| 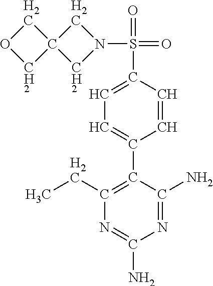 [SMILES: CCc1nc(N)nc(N)c1-c1ccc(S(=O)(=O)N2CC3(COC3)C2)cc1] | WCDD107 | PYR | 5-(4-((2-oxa-6-azaspiro[3.3]heptan- 6-yl)sulfonyl)phenyl)-6- ethylpyrimidine-2,4-diamine | >20 |
| 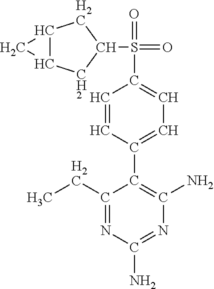 [SMILES: CCc1nc(N)nc(N)c1-c1ccc(S(=O)(=O)C2CC3CC3C2)cc1] | WCDD108 | PYR | 5-(4-(bicyclo[3.1.0]hexan-3- ylsulfonyl)phenyl)-6- ethylpyrimidine-2,4-diamine | 14.87 |
| 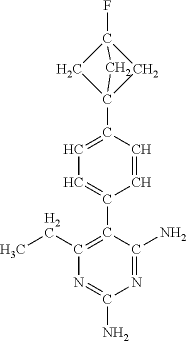 [SMILES: CCc1nc(N)nc(N)c1-c1ccc(C23CC(F)(C2)C3)cc1] | WCDD109 | PYR | 6-ethyl-5-(4-(3- fluorobicyclo[1.1.1]pentan-1- yl)phenyl)pyrimidine-2,4-diamine | |
| 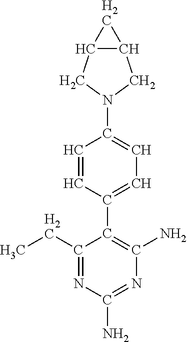 [SMILES: CCc1nc(N)nc(N)c1-c1ccc(N2CC3CC3C2)cc1] | WCDD110 | PYR | 5-(4-(3-azabicyclo[3.1.0]hexan-3- yl)phenyl)-6-ethylpyrimidine-2,4- diamine | >20 |
| 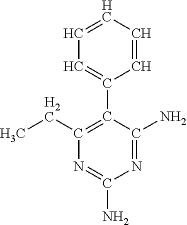 [SMILES: CCc1nc(N)nc(N)c1-c1ccccc1] | WCDD111 | PYR | 6-ethyl-5-phenylpyrimidine-2,4- diamine | 1.175 |
| 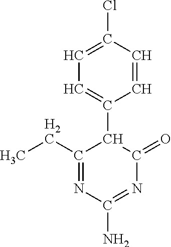 [SMILES: CCC1=NC(N)=NC(=O)C1c1ccc(Cl)cc1] | WCDD112 | PYR | 2-amino-5-(4-chlorophenyl)-6- ethylpyrimidin-4(5H)-one | 13.86 |
| 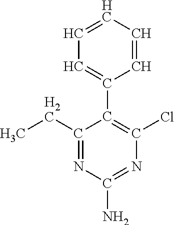 [SMILES: CCc1nc(N)nc(Cl)c1-c1ccccc1] | WCDD113 | PYR | 4-chloro-5-(4-chlorophenyl)-6- ethylpyrimidin-2-amine | >20 |
| 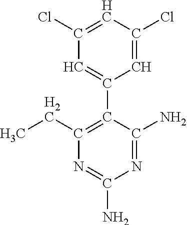 [SMILES: CCc1nc(N)nc(N)c1-c1cc(Cl)cc(Cl)c1] | WCDD114 | WCDD104 | 5-(3,5-dichlorophenyl)-6- ethylpyrimidine-2,4-diamine | 0.108 |
| 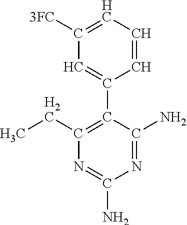 | WCDD115 | WCDD104 | 6-ethyl-5-(3- (trifluoromethyl)phenyl)pyrimidine- 2,4-diamine | 0.057 |
| 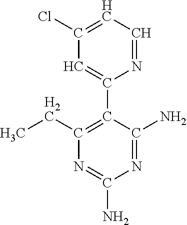 [SMILES: CCc1nc(N)nc(N)c1-c1cc(Cl)ccn1] | WCDD116 | WCDD104 | 5-(4-chloropyridin-2-yl)-6- ethylpyrimidine-2,4-diamine | |
| 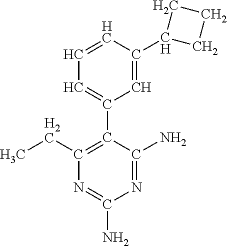 [SMILES: CCc1nc(N)nc(N)c1-c1cccc(C2CCC2)c1] | WCDD117 | WCDD104 | 5-(3-cyclobutylphenyl)-6- ethylpyrimidine-2,4-diamine | |
| 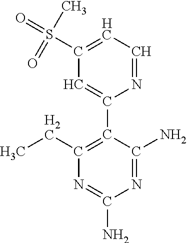 [SMILES: CCc1nc(N)nc(N)c1-c1cc(S(C)(=O)=O)ccn1] | WCDD118 | WCDD104 | 6-ethyl-5-(3- (methylsulfonyl)phenyl)pyrimidine- 2,4-diamine | >5 |
| 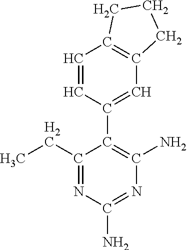 [SMILES: CCc1nc(N)nc(N)c1-c1ccc2c(c1)CCC2] | WCDD119 | WCDD104 | 5-(2,3-dihydro-1H-inden-5-yl)-6- ethylpyrimidine-2,4-diamine | 0.57 |
| 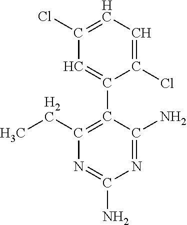 [SMILES: CCc1nc(N)nc(N)c1-c1cc(Cl)ccc1Cl] | WCDD120 | WCDD104 | 5-(2,5-dichlorophenyl)-6- ethylpyrimidine-2,4-diamine | >10 |
| 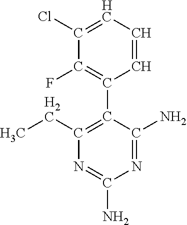 [SMILES: CCc1nc(N)nc(N)c1-c1cccc(Cl)c1F] | WCDD121 | WCDD104 | 5-(3-chloro-2-fluorophenyl)-6- ethylpyrimidine-2,4-diamine | >5 |
| 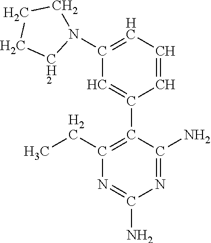 [SMILES: CCc1nc(N)nc(N)c1-c1cccc(N2CCCC2)c1] | WCDD122 | WCDD104 | 6-ethyl-5-(3-(pyrrolidin-1- yl)phenyl)pyrimidine-2,4-diamine | >5 |
| 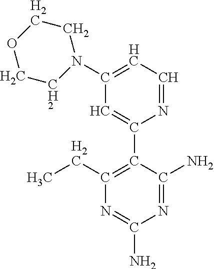 [SMILES: CCc1nc(N)nc(N)c1-c1cc(N2CCOCC2)ccn1] | WCDD123 | WCDD104 | 6-ethyl-5-(3- morpholinophenyl)pyrimidine-2,4- diamine | >20 |
| 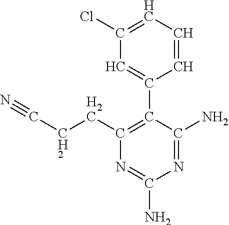 [SMILES: N#CCCc1nc(N)nc(N)c1-c1cccc(Cl)c1] | WCDD124 | WCDD104 | 6-(but-3-yn-1-yl)-5-(3- chlorophenyl)pyrimidine-2,4- diamine | |
| 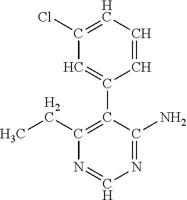 [SMILES: CCc1ncnc(N)c1-c1cccc(Cl)c1] | WCDD125 | WCDD104 | 5-(3-chlorophenyl)-6- ethylpyrimidin-4-amine | >20 |
| 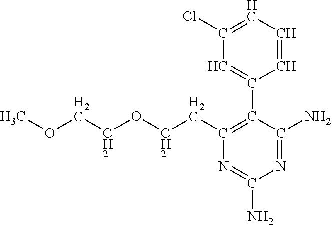 [SMILES: COCCOCCc1nc(N)nc(N)c1-c1cccc(Cl)c1] | WCDD126 | WCDD104 | 5-(3-chlorophenyl)-6-(2-(2- methoxyethoxy)ethyl)pyrimidine- 2,4-diamine | 0.514 |
| 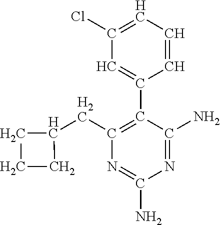 [SMILES: Nc1nc(N)c(-c2cccc(Cl)c2)c(CC2CCC2)n1] | WCDD127 | WCDD104 | 5-(3-chlorophenyl)-6- (cyclobutylmethyl)pyrimidine-2,4- diamine | 0.456 |
| 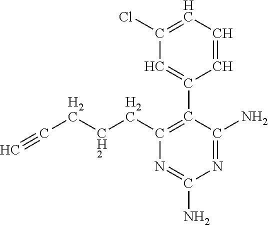 [SMILES: C#CCCCc1nc(N)nc(N)c1-c1cccc(Cl)c1] | WCDD128 | WCDD104 | 5-(3-chlorophenyl)-6-(pent-4-yn-1- yl)pyrirnidine-2,4-diamine | 0.248 |
| 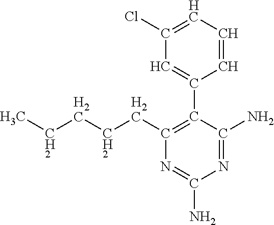 [SMILES: CCCCCc1nc(N)nc(N)c1-c1cccc(Cl)c1] | WCDD129 | WCDD104 | 5-(3-chlorophenyl)-6- pentylpyrimidine-2,4-diamine | |
| 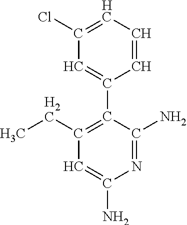 [SMILES: CCc1cc(N)nc(N)c1-c1cccc(Cl)c1] | WCDD130 | WCDD104 | 3-(3-chlorophenyl)-4-ethylpyridine- 2,6-diamine | |
| 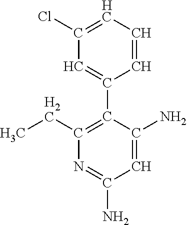 [SMILES: CCc1nc(N)cc(N)c1-c1cccc(Cl)c1] | WCDD131 | WCDD104 | 5-(3-chlorophenyl)-6-ethylpyridine- 2,4-diamine | |
| 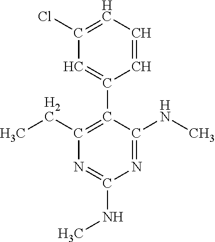 [SMILES: CCc1nc(NC)nc(NC)c1-c1cccc(Cl)c1] | WCDD132 | WCDD104 | 5-(3-chlorophenyl)-6-ethyl-N2,N4- dimethylpyrimidine-2,4-diamine | |
| 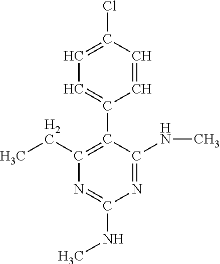 [SMILES: CCc1nc(NC)nc(NC)c1-c1ccc(Cl)cc1] | WCDD133 | PYR | 5-(4-chlorophenyl)-6-ethyl-N2,N4- dimethylpyrimidine-2,4-diamine | |
| 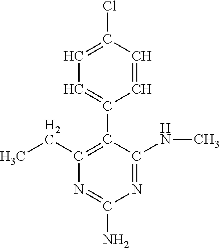 [SMILES: CCc1nc(N)nc(NC)c1-c1ccc(Cl)cc1] | WCDD133a | PYR | 5-(4-chlorophenyl)-6-ethyl-N4- methylpyrimidine-2,4-diamine | |
| 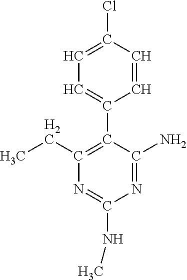 [SMILES: CCc1nc(NC)nc(N)c1-c1ccc(Cl)cc1] | WCDD133b | PYR | 5-(4-chlorophenyl)-6-ethyl-N2- methylpyrimidine-2,4-diamine | |
| 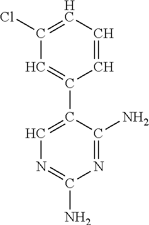 [SMILES: Nc1ncc(-c2cccc(Cl)c2)c(N)n1] | WCDD134 | WCDD104 | 5-(3-chlorophenyl)pyrimidine-2,4- diamine | 1.43 |
| 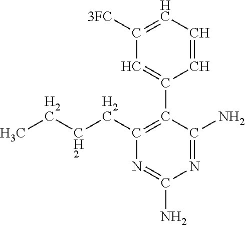 | WCDD135 | WCDD115 | 6-(3-aminopropyl)-5-(3- (trifluoromethyl)phenyl)pyrimidine- 2,4-diamine | |
| 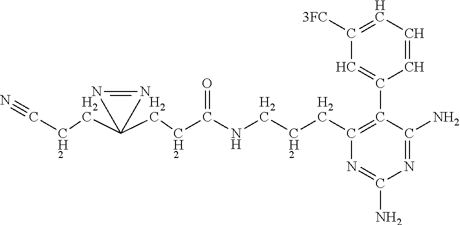 | WCDD136 | WCDD115 | 3-(3-(but-3-yn-1-yl)-3H-diazirin-3- yl)-N-(3-(2,6-diamino-5-(3- (trifluoromethyl)phenyl)pyrimidin- 4-yl)propyl)propanamide | |
| 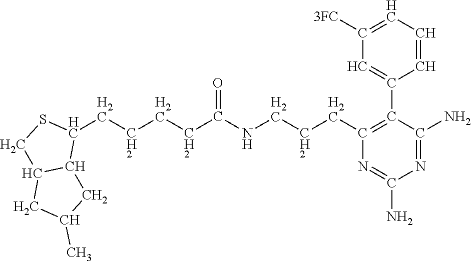 | WCDD137 | WCDD115 | N-(3-(2,6-diamino-5-(3- (trifluoromethyl)phenyl)pyrimidin- 4-yl)propyl)-5-((4R)-2- hydroxyhexahydro-1H-thieno[3,4- d]imidazol-4-yl)pentanamide | |
| 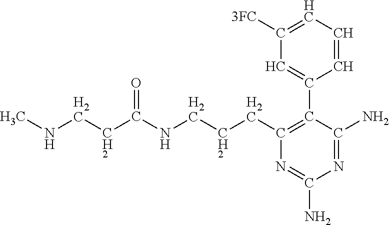 | WCDD138 | WCDD115 | N-(3-(2,6-diamino-5-(3- (trifluoromethyl)phenyl)pyrimidin- 4-yl)propyl)pent-4-enamide | |
| 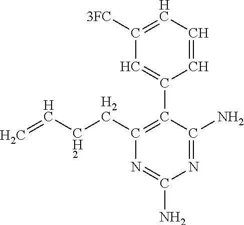 | WCDD139 | WCDD115 | 6-(but-3-en-1-yl)-5-(3- (trifluoromethyl)phenyl)pyrimidine- 2,4-diamine | 0.14 |

What is claimed is:

1. A compound of Formula (I) or a pharmaceutically acceptable salt thereof: 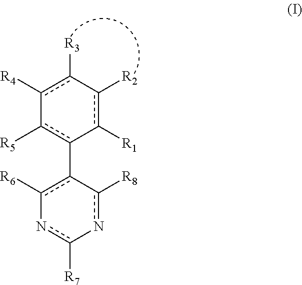
wherein:
R₁, R₂, R₃, R₄, R₅, R₆, R₇, and R₈ are independently selected from the group consisting of amino, amino-C₁₋₁₀ alkyl, bi-cyclo-alkyl-substituted sulfonyl, bi-hetrocyclyl, carboxyl-substituted-C₁₋₁₀ alkyl, carboxyl-substituted- or heterocyclyl-substituted-C₁₋₁₀ alkyl, carboxyl-substituted- or heterobicyclyl-substituted-C₁₋₁₀ alkyl, C₁₋₁₀ alkyl, C₁₋₁₀ alkoxy-substituted C₁₋₁₀ alkyl, C₁₋₁₀ alkylsulfonyl, C₁₋₁₀ cycloalkyl, C₁₋₁₀ heteroclyclyl, C₃₋₁₀ cycloalkyl-substituted sulfonyl, C₃₋₁₀ cycloalkyl-substituted C₁₋₁₀ alkyl, H, halogen, halo-substituted C₁₋₁₀ alkyl, halo-substituted bi-C₃₋₁₀ cycloalkyl, hetro-bi-cyclo-alkyl-substituted sulfonyl, heterocyclyl-substituted C₁₋₁₀ alkyl, oxo- or oxy-substituted C₁₋₁₀ alkyl, and optionally, R2 and R3 are linked with C₁₋₁₀ alkyl.

2. The compound of claim 1, wherein R₁ is selected from the group consisting of H or halogen.

3. The compound of claim 1, wherein R₂ is selected from the group consisting of H, halogen, and C₁₋₁₀ cycloalkyl.

4. The compound of claim 1, wherein R₂ and R₃ are linked with C₁₋₁₀ alkyl.

5. The compound of claim 1, wherein R₃ is selected from the group consisting of H, halogen, halo-substituted C₁₋₁₀ alkyl, C₃₋₁₀ cycloalkyl-substituted sulfonyl, halo-substituted bi-C₃₋₁₀ cycloalkyl, hetro-bi-cyclo-alkyl-substituted sulfonyl or bi-cyclo-alkyl-substituted sulfonyl, and bi-hetrocyclyl.

6. The compound of claim 6, wherein R₃ is H, Cl, cyclopentylsulfonyl, azaspiro[3.3]heptan-6-yl)sulfonyl, bicyclo[3.1.0]hexan-3-ylsulfonyl, 3-fluorobicyclo[1.1.1]pentan-1-yl, or azabicyclo[3.1.0]hexan-3-yl.

7. The compound of claim 1, wherein R₄ is selected from the group consisting of H, halogen, halo-substituted C₁₋₁₀ alkyl, C₁₋₁₀ alkylsulfonyl, and C₁₋₁₀ heterocyclyl.

8. The compound of claim 7, wherein R₄ is H, Cl, trifluoromethyl, methylsulfonyl, pyrrolidin-1-yl, or morpholino.

9. The compound of claim 1, wherein R₅ is selected from the group consisting of H and halogen.

10. The compound of claim 1, wherein R₆ is selected from the group consisting of H, aminoC₁₋₁₀alkyl, C₁₋₁₀ alkyl, C₁₋₁₀ alkoxy-substituted C₁₋₁₀ alkyl, oxy-substituted C₁₋₁₀ alkyl, C₃₋₁₀ cycloalkyl-substituted C₁₋₁₀ alkyl, carboxyl-substituted C₁₋₁₀ alkyl, heterocyclyl-substituted C₁₋₁₀ alkyl, carboxyl-substituted- or heterocyclyl-substituted-C₁₋₁₀ alkyl, and carboxyl-substituted- or heterobicyclyl-substituted-C₁₋₁₀ alkyl.

11. The compound of claim 10, wherein R₆ is H, ethyl, but-3-yn-1-yl, methoxyethoxy ethyl, cyclobutylmethyl, pent-4-yn-1-yl, pentyl, but-3-en-1-yl, aminopropyl, 3-(3-(3-(but-3-yn-1-yl)-3H-diazirin-3-yl)propanamido)propanyl, 3-(5-((4R)-2-hydroxyhexahydro-1H-thieno[3,4-d]imidazol-4-yl)pentanamido)propanyl, or N-(3λ3-propyl)pent-4-enamidyl.

12. The compound of claim 1, wherein R₇ is selected from the group consisting of amino and H.

13. The compound of claim 1, wherein R₈ is selected from the group consisting of amino, H, halogen, and oxo.

14. The compound of claim 13, wherein R₈ is H, amino, oxo, Cl, or methyl amino.

15. The compound of claim 1, wherein the compound is not pyrimethamine or 5-(3-chlorophenyl)-6-ethylpyrimidine-2,4-diamine (WCDD104).

16. The compound of claim 1, wherein the compound is selected from the group consisting of

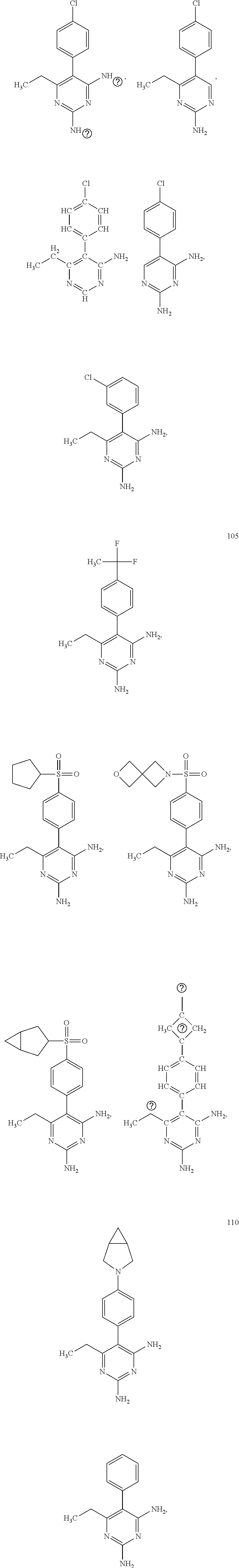

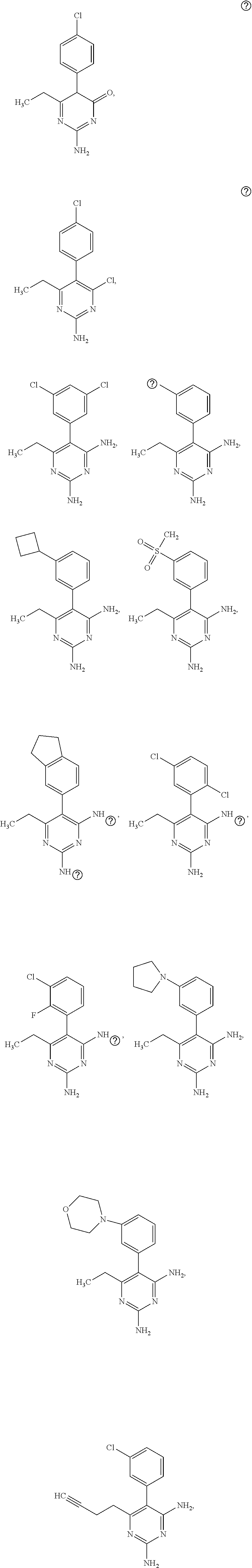

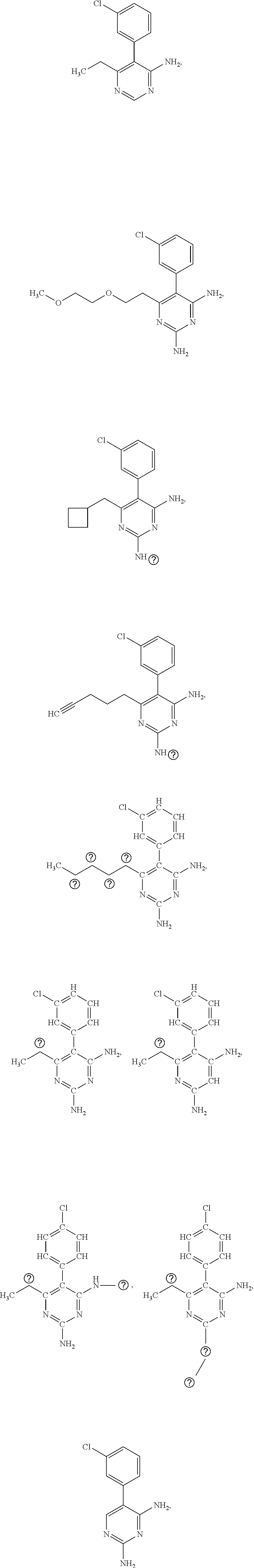

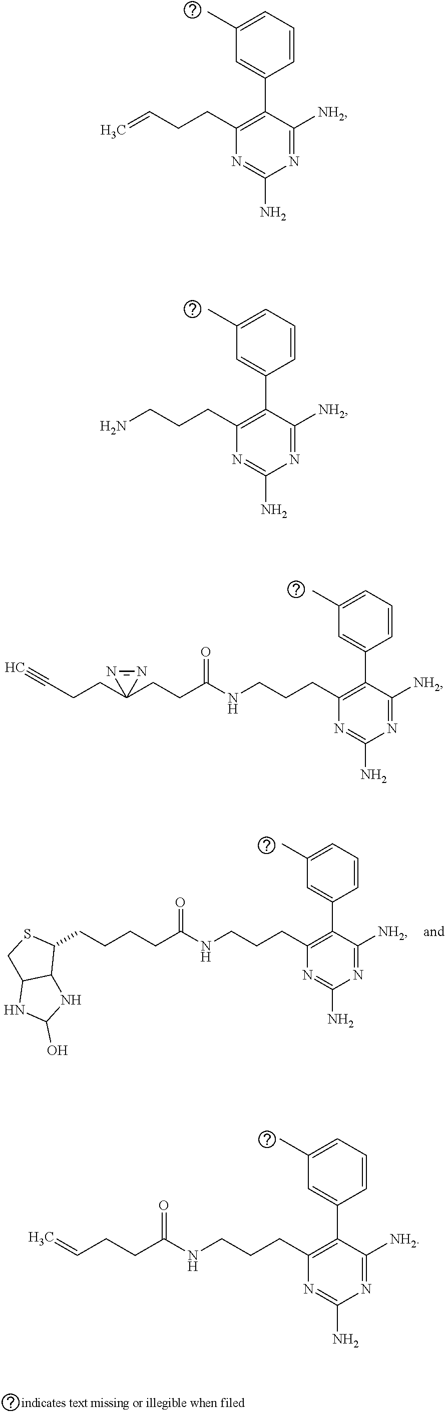

17. A method of inhibiting or suppressing NRF2 activity or function in a subject comprising:
administering to the subject an effective amount of a composition comprising a compound of claim 1, wherein NRF2 expression or activity is reduced in a cell of the subject relative to a cell in the subject prior to administration of the composition.

18. The method of claim 17, wherein the amount effective to inhibit NRF2 activity or function is an amount that decreases NRF2 mRNA, NRF2 protein abundance, or expression of NFR2-mediated downstream targets.

19. A method of treating a subject with cancer, the method comprising:
administering to the subject an effective amount of a composition comprising a compound of claim 1, wherein NRF2 expression or activity is reduced in a cancer cell in the subject relative to a cancer cell in the subject prior to administration of the composition.

20. The method of claim 19, wherein the method further comprises administrating chemotherapy or radiation, separately or together to the subject.
